SEQUENCE LISTING (10 sequences)

SEQ ID NO: 1 - DNA, 10 bp, Artificial
  10-mer; peptide nucleic acid PNA10 with a polyamide backbone instead of a sugar-phosphate backbone
atcttctagt

SEQ ID NO: 2 - DNA, 13 bp, Artificial
  13-mer; peptide nucleic acid PNA13 with a polyamide backbone instead of a sugar-phosphate backbone
ttccccttcccaa

SEQ ID NO: 3 - DNA, 13 bp, Artificial
  13 mer; DNAcomp
ttgggaaggggaa

SEQ ID NO: 4 - DNA, 13 bp, Artificial
  13 mer; DNAm1
ttgggagggggaa

SEQ ID NO: 5 - DNA, 20 bp, Artificial
  20-mer; peptide nucleic acid PNA20 with a polyamide backbone instead of a sugar-phosphate backbone
ttgcactgtactcctcttga

SEQ ID NO: 6 - DNA, 20 bp, Artificial
  20 mer; DNAcomp
tcaagaggagtacagtgcaa

SEQ ID NO: 7 - DNA, 22 bp, Artificial
  22-mer; peptide nucleic acid PNA22 with a polyamide backbone instead of a sugar-phosphate backbone
aaccacacaacctactacctca

SEQ ID NO: 8 - DNA, 22 bp, Artificial
  22 mer; DNAcomp
tgaggtagtaggttgtgtggtt

SEQ ID NO: 9 - DNA, 22 bp, Artificial
  22 mer; DNAnc
caaaacaaagatctacatggat

SEQ ID NO: 10 - DNA, 22 bp, Artificial
  22 mer; DNAm1
tgaggtagtaagttgtgtggtt

(19) United States
(12) Patent Application Publication
(10) Pub. No.: US 2012/0070909 A1
(43) Pub. Date: Mar. 22, 2012

(54) LABEL-FREE METHOD FOR DETECTING PRESENCE OR ABSENCE OF NUCLEIC ACIDS

(76) Inventors: Xiaodi Su, Singapore (SG); Roejarek Kanjanawarut, Singapore (SG)

(21) Appl. No.: 13/201,450

(22) PCT Filed: Feb. 10, 2010
(86) PCT No.: PCT/SG10/00047

§ 371 Date: Nov. 17, 2011

Related U.S. Application Data
(60) Provisional application No. 61/152,372, filed on Feb. 13, 2009.

(51) Int. Cl. G01N 33/53; B82Y 15/00

(52) U.S. Cl. 436/501; 977/773; 977/920

(57) ABSTRACT

The present invention is directed to a method of detecting presence or absence of a target nucleic acid using negatively charged metallic nanoparticles dissolved in a solution. A nucleic acid probe with a substantially neutral net charge is used for detecting the target nucleic acid. The present invention is also directed to a kit including at least one nucleic acid probe with a substantially neutral net charge and at least one type of negatively charged metallic nanoparticles for carrying out the method.

### CROSS-REFERENCE TO RELATED APPLICATIONS

This application claims the benefit of priority of U.S. provisional application No. 61/152,372, filed Feb. 13, 2009, the contents of it being hereby incorporated by reference in its entirety for all purposes.

### FIELD OF THE INVENTION

The present invention refers to the field of biochemistry and in particular to technologies concerning the detection of nucleic acids.

### BACKGROUND OF THE INVENTION

Detection of specific oligonucleotide sequences has important applications in medical research, clinical diagnosis, food and drug industry, and environmental monitoring.

Most assays identify specific sequence through hybridization of an immobilized probe to the target analyte after the latter has been modified with a covalently linked label such as a fluorescent or radioactive tag. Oligonucleotide detection schemes that avoid analyte tagging, such as surface plasmon resonance, imaging ellipsometry, and sandwich assays using chemically functionalized gold nanoparticles, have also been invented. Gold nanoparticles covalently functionalized with DNA sequences to bind specific target DNA sequences for oligonucleotide sensing have also been used.

Present assays are dominated by chip-based methodologies that detect DNA sequences through hybridization of an immobilized probe to the target analyte that is labeled with fluorescent, radioactive and chemiluminescent molecular probes. Other biosensor approaches, such as surface plasmon resonance, imaging ellipsometry, quartz crystal microbalance etc, eliminate the use of biohazard labels but still require complex surface attachment chemistry for probe immobilization and expensive equipments for detection. In addition, the hybridization to sterically constrained probes on surface suffers from slow response and low efficiency. All these disadvantages (using complex labels, surface functionalization chemistry, low hybridization efficiency, and the use of expensive and bulky measurement instrumentation) render the chip-based methodologies less amendable for fast and robust DNA detection for point-of-care applications and for on-site applications.

Therefore, there is a need for a nucleic acid recognition assay that is fast and requires no modification of the probe or target nucleic acid.

### SUMMARY OF THE INVENTION

In a first aspect the present invention is directed to a method of detecting presence or absence of a target nucleic acid, wherein the method comprises:

providing a nucleic acid probe with a substantially neutral net charge and contacting the nucleic acid probe with a sample which is suspected to comprise the target nucleic acid to result in a first mixture; wherein the nucleic acid probe is substantially complementary to the target nucleic acid;

contacting the first mixture with negatively charged metallic nanoparticles to result in a second mixture;

determining whether the target nucleic acid is comprised in the second mixture or not.

In a second aspect the present invention is directed to a method of detecting presence or absence of a target nucleic acid, wherein the method comprises:

incubate negatively charged metallic nanoparticles with a sample which is suspected to comprise the target nucleic acid to form a first mixture;

contacting the first mixture with a nucleic acid probe with a substantially neutral net charge to result in a second mixture; wherein the nucleic acid probe is substantially complementary to the target nucleic acid;

determining whether the target nucleic acid is comprised in the second mixture or not.

In a further aspect the present invention is directed to a kit for detecting presence or absence of a target nucleic acid in a sample; wherein the kit comprises:

at least one nucleic acid probe with a substantially neutral net charge;

at least one type of negatively charged metallic nanoparticles.

In still another aspect the present invention is direct to the use of at least one type of negatively charged metallic nanoparticle in a method of the present invention or in a kit of the present invention.

In still another aspect the present invention is direct to the use of at least one nucleic acid probe with a substantially neutral net charge in a method of the present invention or in a kit of the present invention.

### BRIEF DESCRIPTION OF THE DRAWINGS

The invention will be better understood with reference to the detailed description when considered in conjunction with the non-limiting examples and the accompanying drawings, in which:

FIG. 1(A) illustrates the detection principle for the target nucleic acid according to the first aspect of the present invention. Negatively charged metallic particles are well dispersed due to the repulsion of the charges at the surface of the metallic particles and thus appear in a first color (starting point) (different colors are shown using different shades for the particles). Contacting the negatively charged metallic particles dispersed in a solution with different kinds of nucleic acids results in a color change due to different degrees of aggregation with the different nucleic acids. The degree of aggregation depends on how well the negative charges at the surface of the metallic particles are shielded by the different kinds of nucleic acids. For example, in case a nucleic acid probe with a substantially neutral net charge, such as PNA is added alone to the negatively charged metallic particles (left pathway) the PNA binds to the surface of the negatively charged metallic particles which leads to a substantial shielding of the negative charges and thus to an aggregation of the metallic particles. Aggregation of the metallic particles takes place because the negative surface charges of the metallic particles are shielded by the nucleic acid probe with the substantially neutral net charge bound to the surface of the metallic particles. Due to the aggregation of the metallic particles the absorption of the metallic particles changes which can be measured. In a detection assay in which the nucleic acid probe with a substantially neutral net charge is at first contacted with a sample which for example comprises the substantially complementary target nucleic acid (middle pathway), the nucleic acid probe with a substantially neutral net charge, such as PNA, anneals in the hybridization step following a potential previous heating step to the respective target nucleic acid. Annealing of the nucleic acid probe with the substantially neutral net charge, such as PNA, with the target sequence leads to the formation of a double helix between them (PNA-DNA-complex in FIG. 1). This double helix now binds to the negatively charged metallic particles but does not cause observable aggregation of the metallic particles as it does if only the nucleic acid probe with the substantially neutral net charge is contacted with the negatively charged metallic particles. This difference leads to a different absorption which can be measured with a photometer or is even visible to the naked eye. Also, the degree of aggregation of the negatively charged metallic particles is different in case the nucleic acid probe with the substantially neutral net charge is mixed with a sample which does not comprise the target nucleic acid but other nucleic acids which are not complementary to the nucleic acid probe (right pathway). Again the degree of aggregation of the negatively charged metallic particles is different thus leading to a different absorption. This system is even usable to differentiate between fully complementary target nucleic acids and target nucleic acids which comprise a single mutation in its nucleic acid sequence compared to the nucleic acid probe with the substantially neutral net charge as has been shown in the examples referred to herein (see also FIG. 16).

FIG. 1(B) illustrates an embodiment in which the negatively charged metallic particles are contacted at first with the sample which is suspected to comprise the complementary (target) nucleic acid. In this aspect the detection is based on the finding that the nucleic acid probe with a substantially neutral net charge has a higher binding affinity to the metallic particles than any other nucleic acid which might be comprised in the sample, such as the complementary (target) nucleic acid or any other nucleic acid which is not the target nucleic acid (non-complementary nucleic acid). Thus, complementary (target) or non-complementary nucleic acid (1) which after mixing with the metallic particles binds to the metallic particles is at least partly displaced after mixing with a nucleic acid probe with a substantially neutral net charge (3). Mixing of the metallic particles with the different molecules, i.e. complementary (target) nucleic acid, non-complementary nucleic acid, and nucleic acid probe, results in a different aggregation of the metallic particles which can be detected. In case the metallic particles (2) which have already been contacted with a sample which comprises the complementary target nucleic acid, the nucleic acid probe (3) hybridizes with the target nucleic acid bound to the metallic particles and binds to the metallic particles which results in no detectable aggregation (4). In case the nucleic acid which was mixed together with the metallic particles (2) was not the complementary (target) nucleic acid but the non-complementary nucleic acid, the nucleic acid probe with the substantially neutral net charge added (3) to the first mixture partly displaces the non-complementary nucleic acid already bound to the metallic particles (5). This leads to an aggregation which can be detected and leads to a degree of differentiation which is distinguishable from the situation in which the nucleic acid probe is mixed with a first mixture which included the complementary (target) nucleic acid (4). With respect to such an embodiment please see also the exemplary embodiment in the experimental section with the title: “Detection of pre-annealed PNA-DNA complex with single-base-mismatch sensitivity using AuNPs and AgNPs”

FIG. 2 shows an UV-vis adsorption spectra of AuNPs and AuNPs exposed to PNA₂₀/DNA mixture (DNA_{nc}). PNA₂₀ concentration is 1 μM. The stability of AuNPs solutions exposed to mixtures of the 20 mer PNA₂₀ (1 μM) and ssDNA of same sequence (no hybridization occurs) at ssDNA(DNA_{nc})/PNA ratio of 0, 5, and 10 is shown. PNA-induced particle aggregation remains detectable when ssDNA is in great excess

FIG. 3 shows an overlay of 8 scans of UV-vis adsorption spectrum of AuNPs dispensed in different wells. Insets are a close look at wavelengths around 520 nm and 600 nm.

FIG. 4 shows an overlay of 8 scans of UV-vis adsorption spectrum of AgNPs dispensed in different wells. Insets are a close look at wavelengths around 400 nm and 600 nm.

FIG. 5 shows PNA-induced AuNPs aggregation. UV-vis adsorption spectra of AuNPs (13 nm in diameter) exposed to (A) 13-mer PNA₁₃ and (B) 22-mer PNA₂₂ at different concentrations. (C) Plots of aggregation degree (measured as A600/A520) versus PNA concentration for the 10-, 13-, 20-, and 22-mer PNAs.

FIG. 6 illustrates that PNA has a higher affinity to AuNPs than its ssDNA counterpart. UV-vis adsorption spectra of bare AuNPs and AuNPs after 10 min incubation with a mixture of PNA₂₂ (200 nM) and ssDNA of same sequence (termed as noncDNA_{nc}) at DNA/PNA ratio of 0, 5, and 10.

FIG. 7 illustrates that PNA-DNA hybridization disrupts particle aggregation. UV-vis adsorption spectra of AuNPs solutions after 10 min incubation with PNA₂₂ (200 nM) annealed with its complementary DNA_{comp} at 0, 20, 40, 100, and 160 nM. The inset is the plot of aggregation degree (measured as A600/A520) versus DNA/PNA ratio.

FIG. 8 shows stabilization effects of different nucleic acids to AuNPs. Photographs of AuNPs solutions taken after 10 min incubation with ssDNA, dsDNA, and PNA-DNA complex (20-mer and 13-mer samples), without and with addition of NaCl (0.05, 0.1, and 0.15 M) at the end of incubation are shown. The respective UV-vis adsorption spectra are for solutions with 0.15 M NaCl. The final concentration of all nucleic acids samples is 1 μM.

FIG. 9 illustrates stabilization effects of different nucleic acids to AgNPs. (A) Photographs of AgNPs solutions recorded after 10 min incubation with ssDNA, dsDNA, PNA-DNA complex, and PNA (13-mer samples), without and with addition of NaCl (final concentration 0.05, 0.1, and 0.15 M) at the end of incubation. (B) and (C) are corresponding UV-vis adsorption spectra before and after addition of NaCl (0.15 M), respectively. The final concentration of all nucleic acids is 1 μM. (BY=bright yellow; LY=light yellow; VLY=very light yellow; B=brownish; LB=light brown; VLB=very light brownish; T=transparent;)

FIG. 10 illustrates single-base-mismatch discrimination in pre-annealed PNA-DNA complexes. UV-vis adsorption spectra of (A) AuNPs and (B) AgNPs after 10 min incubation with PNA₁₃ pre-annealed with complementary (DNA_{comp}) and single-base-mismatch (DNAₘ₁) targets, without and with addition of NaCl (0-0.4 M) at the end of incubation. Final nucleic acid concentration is 1 μM.

FIG. 11 illustrates the results of the colorimetric detection of PNA hybridization with DNA in DNAIAuNPs mixtures. Color photographs of AuNPs and AuNPs mixed with DNA_{comp}, DNAₘ₁, and DNA_{nc} (final concentration 1 μM) of (A) 13-mer and (B) 22-mer samples, before and after addition of respective PNA (final concentration 1 μM). NaCl is further added into the 22-mer sample wells.

FIG. 12 shows the results of SPR measurement of target DNA hybridization to PNA₁₃ immobilized surface in (left) 1 mM and (right) 0.1 mM PBS buffer at room temperature.

FIG. 13 illustrates AuNPs-based colorimetric detection of specific DNA sequence, using AuNPs' intrinsic stability. Photography and corresponding UV-vis spectra of (A) bare AuNP, (B) AuNP+PNA, (C) AuNP+PNA-DNA hybrid, and (D) AuNP+PNA/DNA mixture.

FIG. 14 illustrates AuNPs-based colorimetric detection of specific DNA sequence, using AuNPs' stability against salt. Photography and corresponding UV-vis spectra of (A) bare AuNP, (B) AuNP+PNA, (C) AuNP+PNA-DNA hybrid, and (D) AuNP+PNA/DNA mixture exposed to 0.1 M NaCl.

FIG. 15 illustrates AuNPs-based colorimetric detection of target DNA using DNA probes. Photography and corresponding UV-vis spectra of (A) AuNP+ssDNA and (B) AuNP+dsDNA (e.g. DNA hybrid) exposed to 0.1 M NaCl.

FIG. 16 shows a schematic diagram for scheme used for one-component assay for detection of single-base-mismatch in a nucleic acid using negatively charged metallic particles. To further discriminate between target nucleic acids which are fully complementary (Comp) to the nucleic acid probe and target nucleic acids which are not fully complementary, such as nucleic acids comprising single-base mismatch in one position of the target sequence (M1), it is possible to alter the hybridization conditions. For example, the addition of monovalent cations, such as sodium chloride (NaCl) or divalent cations to the hybridization mixture or second mixture can further change the degree of metallic particle aggregation depending on whether a fully complementary or single-base mismatch target nucleic acid is comprised in the test sample. As has been demonstrated in the experimental section, such differentiation is possible even at room temperature, i.e. without a further step of increasing the temperature to increase hybridization stringency.

FIG. 17 AuNPs-based colorimetric detection of single-base-mismatch. (A) bare AuNP exposed to PNA probe (B) AuNP exposed to PNA-fully complementary DNA complex (C) AuNP exposed to PNA-single-base-mismatch DNA complex.

FIG. 18 illustrates AgNPs-based colorimetric detection of specific DNA sequence, using the particles' intrinsic stability. Photography and corresponding UV-vis spectra of (A) AgNP+PNA and (B) AgNP+PNA-DNA hybrid, without addition of NaCl.

FIG. 19 AgNPs-based colorimetric detection of single-base-mismatch using particles' stability against salt. Photography and corresponding UV-vis spectra of (A) AgNP+PNA, (B) AgNP+PNA-DNA complementary hybrid, and (C) AgNP+PNA-DNA SBM with the addition of NaCl.

FIG. 20 shows UV-vis spectra of AuNPs, AgNPs, and the mixture of the two.

FIG. 21 shows two-component detection of single-base-mismatch in DNA, using mixed AuNPs and AgNPs, in the presence of 0.1 M (up) and 0.2 M (bottom) NaCl.

### DETAILED DESCRIPTION OF THE PRESENT INVENTION

The present invention is based on the fact that the absorption, such as the UV-vis absorption, of dispersed metallic nanoparticles (NPs) in a solution changes when the nanoparticles are aggregated. The aggregation degree is influenced by the exposure to different nucleic acids. This fact can be used to create a detection assay. For such a detection assay nucleic acids with a substantially neutral net charge are used as probe to detect the hybridization of negatively charged target nucleic acids, such as DNA and RNA. The nucleic acid probe with the substantially neutral net charge as such, a hybrid of a target nucleic acid with the nucleic acid probe formed by complementary binding of target nucleic acid with nucleic acid probe, and non hybridized mixtures of nucleic acid probe and target nucleic acid (due to no matching in the nucleic acid sequence and thus no complementary binding) exert different effects on the metallic nanoparticles behavior due to their different ability to interact with the metallic nanoparticles.

Thus, in a first aspect the present invention is directed to a method of detecting presence or absence of a target nucleic acid in a solution. This method does not require that any of the molecules involved in the method is attached to a solid phase. All components used can be dispersed in a liquid or aqueous solution. The method comprises:

providing a nucleic acid probe with a substantially neutral net charge and contacting the nucleic acid probe with a sample which is suspected to comprise the target nucleic acid to result in a first mixture; wherein the nucleic acid probe is substantially complementary to the target nucleic acid;

contacting the first mixture with negatively charged metallic particles to result in a second mixture;

determining whether the target nucleic acid is comprised in the second mixture or not.

In one embodiment the target nucleic acid is a label-free target nucleic acid or unlabeled target nucleic acid. With this method the presence and absence of a specific target nucleic acid sequence down to a single-base mutation in the nucleic acid can be determined rapidly by measuring for example the absorbance or change in particle size caused by aggregation in the particle solution. This method is fast and efficient since hybridization is completely separated from detection, thus eliminating sterical constrains of surface-bound probes or labeled probes which slow down hybridization and the entire detection process dramatically. This method is also easier to use because it does not require a modification of the nucleic acid probe or target nucleic acid (labeling with fluorescence or radioactive marker, coupling to enzymes, etc.) for detection.

The term “nucleic acid molecule” as used herein refers to any nucleic acid in any possible configuration, such as single stranded, double stranded or a combination thereof. Nucleic acids include for instance DNA molecules, RNA molecules, analogues of the DNA or RNA generated using nucleotide analogues or using nucleic acid chemistry, locked nucleic acid molecules (LNA), PNA molecules and tecto-RNA molecules (e.g. Liu, B., et al., J. Am. Chem. Soc. (2004) 126, 4076-4077). An LNA molecule has a modified RNA backbone with a methylene bridge between C4′ and O2′, which locks the furanose ring in a N-type configuration, providing the respective molecule with a higher duplex stability and nuclease resistance. Unlike a PNA molecule an LNA molecule has a charged backbone. DNA or RNA may be of genomic or synthetic origin and may be single or double stranded. Such nucleic acid can be e.g. mRNA, cRNA, synthetic RNA, genomic DNA, cDNA synthetic DNA, a copolymer of DNA and RNA, oligonucleotides, micro RNA having a length of between about 21 to 23 nucleotides etc. A respective nucleic acid may furthermore contain non-natural nucleotide analogues.

Many nucleotide analogues are known and can be present and/or used in the methods of the invention. A nucleotide analogue is a nucleotide containing a modification at for instance the base, sugar, or phosphate moieties. As an illustrative example, a substitution of 2′-OH residues of siRNA with 2′F, 2′O-Me or 2′H residues is known to improve the in vivo stability of the respective RNA. Modifications at the base moiety include natural and synthetic modifications of A, C, G, and T/U, different purine or pyrimidine bases, such as uracil-5-yl, hypoxanthin-9-yl, and 2-aminoadenin-9-yl, as well as non-purine or non-pyrimidine nucleotide bases. Other nucleotide analogues serve as universal bases. Universal bases include 3-nitropyrrole and 5-nitroindole. Universal bases are able to form a base pair with any other base. Base modifications often can be combined with for example a sugar modification, such as for instance 2′-O-methoxyethyl, e.g. to achieve unique properties such as increased duplex stability.

The “nucleic acid probe with a substantially neutral net charge” refers in general to a nucleic acid which comprises instead of the normal negatively charged ribose- or deoxyribose-phosphodiester backbone a backbone which is not charged (neutral). With ‘substantially neutral’ it is meant that the nucleic acid probe can include a nucleotide with a charged group, such as a negative group or a positive group as long as this does not influence the binding capability of the nucleic acid probe to the metallic particles or in other words as long as the positive or negative charges are not sufficient to balance the shielded negative charges of the metallic particles.

For example, a nucleic acid probe with a substantially neutral net charge could comprise glutamic acid and/or aspartic acid to introduce negative charges to PNA without affecting its ability to bind to the metallic nanoparticles in a more stable manner than nucleic acids, such as DNA or RNA. Also, a nucleic acid probe with a substantially neutral net charge modified with lysine, which has a positive charge, could also be used without affecting the functionality of the nucleic acid probe referred to herein. For example, a PNA used as nucleic acid probe and carrying a positive charge was shown to bind even more effectively to metallic nanoparticles, such as gold nanoparticles and such a modified nucleic acid probe can cause even more aggregation of the metallic nanoparticles. Thus the sensitivity using slightly positively charged nucleic acid probes, such as PNA can be higher.

In one embodiment, the nucleic acid probe with a substantially neutral net charge is not a single stranded DNA (ssDNA).

In one embodiment a PNA probe is used as nucleic acid probe with a substantially neutral net charge. It was found that for example PNA binds more effectively to the metallic nanoparticles than DNA thus making PNA a suitable tool for use in the method of the present invention.

A peptide nucleic acid (PNA) used in the present invention is a molecule which is similar for example to DNA or RNA in that it comprises nucleobases. However, instead of a negatively charged phosphodiester backbone to which the nucleobases are bound it comprises a pseudo-peptide backbone. In more detail, in a PNA the purine (A, G) and pyrimidine (C, T, U) bases are attached to the backbone through methylene carbonyl linkages. Unlike DNA, RNA, DNA analogs or RNA analogs, PNAs do not contain any (pentose) sugar moieties or phosphate groups.

By convention, PNAs are depicted like peptides, with the N-terminus at the first (left) position and the C-terminus at the right. Besides the obvious structural difference, PNA is set apart from DNA or RNA in that the backbone of PNA is acyclic, achiral and in case of the present invention substantially neutral. PNAs can bind to complementary nucleic acids in both antiparallel and parallel orientation. However, the antiparallel orientation is strongly preferred, and the parallel duplex has been shown to have a different structure. In an embodiment of the present invention PNA/nucleic acid duplexes refer to the antiparallel complex. The Watson Crick base pairing rules are strictly observed in hybrids of PNA and nucleic acids.

Thus, PNA is capable of hybridising at least complementary and substantially complementary nucleic acid strands, just as e.g. DNA or RNA, to which PNA is considered a structural mimic. Since a PNA molecule has a neutral backbone, hybridization with target nucleic acids is furthermore not affected by the interstrand negative charge electrostatic repulsions. The binding of PNA to DNA and RNA targets is accordingly stronger than the binding of DNA to DNA or RNA to RNA. PNA/DNA or PNA/RNA hybrids thus show a very stable duplex formation even at a relatively high temperature, and a very high binding affinity. Additionally, the absence of a repetitively charged backbone also prevents a PNA molecule from binding to proteins that normally have an affinity to polyanions. Hence, the use of PNA avoids a major source of non-specific interactions.

As mentioned, a PNA molecule is a synthetic nucleic acid analogue with a pseudopeptide backbone in which the phosphodiester backbone present in e.g. DNA or RNA is replaced by repetitive units of short aliphatic moieties with an amino end and a carboxylic end, forming an amide bond in the oligomer or polymer. To the short aliphatic moieties of the backbone, nucleobases, usually purine and pyrimidine bases, are attached via a side chain, generally a methyl carbonyl linker. In a PNA molecule any desired nucleobase, including purine and pyrimidine bases, may be used. Examples of suitable purine and pyrimidine bases include, but are not limited to, cytosine, 5-methylcytosine, guanine, adenine, thymine, uracil, 5,6-dihydrouracil, hypoxanthine, xanthine, ribothymine, 7-methylguanine or 7-isobutylguanine. A number of further suitable illustrative nucleobases that may also be termed “modified” nucleobases have recently been reviewed by Wojciechowski & Hudson (Current Topics in Medicinal Chemistry (2007) 7, 667-679). These include inter alia 4-(1,2,4-triazolypthymine, 5-alkynyluracil, 5-iodouracil, thiouracil, 5-(propargyl alcohol)uracil, iso-cytosine, pseudoisocytosine, 5-(ferrocenylpropargylcarboxamide)uracil, N6-alkyladenine, N7-xanthine, 3-nitropyrrole, 6-thioguanine, phenoxazine, 2-aminopurine or 2,6-diaminopurine.

The classical and most used PNA consists of repeating N-(2-aminoethyl)-glycine units linked by amide bonds. However, the backbone of this PNA lends itself to further chemical modifications. For example, the glycine units in the N-(2-aminoethyl)-glycine unit can be easily substituted either partly or completely by all naturally occurring amino acids. In one embodiment of the present invention it is preferred that the amino acids which are used to replace glycine also have a neutral net charge, such as alanine, valine, leucine, isoleucine, proline, phenylalanine, methionine, tryptophan, cysteine. Other modifications of the backbone of PNAs which are known in the art can also be used herein.

In one embodiment, the nucleic acid probe used in the method of the present invention has a length of between about 8 to 50 nucleosides or between about 10 to 30 nucleosides. In another embodiment the nucleic acid probe has a length of between about 8 to 25 nucleosides or between about 10 to 25 nucleosides. In examples referred to herein the nucleic acid probe has a length of 10, 13, 20 and 22 nucleosides.

The target nucleic acid can be any nucleic acid which can be comprised in a sample to be tested. This nucleic acid can be a naturally occurring nucleic acid or a modified nucleic acid comprising any of the nucleic acid modifications referred to above. In one embodiment, the target nucleic acid is DNA or RNA, such as micro RNA, or derivatives of such nucleic acids. The target nucleic acid can be a single stranded or double stranded nucleic acid. The target nucleic acid can but does not need to be labeled to be detectable with the method of the present invention. A “label” or “detectable label” is a moiety that can be sensed. Such labels can be, without limitation, fluorophores (e.g., fluorescein (FITC), phycoerythrin, rhodamine), chemical dyes, or compounds that are radioactive, chemoluminescent, magnetic, paramagnetic, promagnetic, quantum dots or enzymes that yield a product that may be colored upon reaction with a specific substrate, chemoluminescent, or magnetic. The signal generated by a label is generally detectable by any suitable means, including spectroscopic, photochemical, biochemical, immunochemical, electrical, optical or chemical means.

The method of the present invention cannot only be used to detect the presence or absence of the target nucleic acid but also to quantify the amount of target nucleic acid present. Depending on the amount of target nucleic acid present in a sample compared to another sample in which the target nucleic acid is comprised in a different amount, the degree of metallic particle aggregation will vary. A change of the degree of metallic particle aggregation will change for example the absorbance of the solution or the size of the metallic particle aggregate. This difference can be correlated to different amounts of target nucleic acid present in the sample. To determine the exact amount of target nucleic acid present in the sample the results obtained from a sample can be compared with the results of a sample comprising a known amount of target nucleic acid. Methods to carry out such correlations are known in the art and include for example the generation of calibration curves obtained by measuring samples including a known amount of a target nucleic acid or different target nucleic acids.

In one embodiment, multiple target nucleic acids are measured in one sample by providing multiple nucleic acid probes which are specific for different target nucleic acids. In one embodiment two or three different target nucleic acids are measured in parallel in one sample. The resulting optical property of the metallic particle aggregates changes in case more than one target nucleic acid is present and thus allows determining whether only one target nucleic acid is present or more than one. In one embodiment, different metallic particles are used which have different optical properties and thus can result in different absorbance signals which can be easily differentiated by spectroscopic methods known in the art.

The degree of aggregation of the metallic nanoparticles referred to herein changes upon binding of a nucleic acid. The degree of aggregation depends on the kind of nucleic acid that binds. Aggregation changes for example the absorbance or the size caused by aggregation of multiple single nanoparticles. These changes can be measured using standard microscopic or spectroscopic methods.

In one embodiment, to enhance the measurability of these changes the metallic nanoparticles are plasmonic nanoparticles. Plasmonic nanoparticles refer to metallic nanoparticles whose surface Plasmon resonance frequency depends on the nanoparticle size, shape and composition. Light incident on plasmonic nanoparticles induces the conduction electrons in them to oscillate collectively with a certain resonant frequency. This resonant frequency changes in case the metallic nanoparticles aggregate and thus can be measured, e.g. with optical methods.

For example, a single 80-nm silver nanosphere scatters 445-nm blue light with a scattering cross-section of 3×10⁻² μm². The shape of the nanoparticle extinction and scattering spectra, and in particular the peak wavelength λₘₐₓ depends on nanoparticle composition, size, shape, orientation and local dielectric environment.

Manufacture of such plasmonic nanoparticles is known in the art (see e.g. Grabar, K. C., Freeman, R. G., et al., 1995, Anal. Chem., vol. 67, pp. 735). Such nanoparticles can be obtained in any known shape. For example, in one embodiment the nanoparticles have a shape which is selected from the group consisting of a nanosphere, a nanocube, a nanorod, a nanotube, a nanostar, a nanocrescent, a nanoplate and mixtures thereof. With mixtures thereof it is meant that the nanoparticles used can include nanoparticles having not only a tubular shape but also nanoparticles having a plate like shape. In one embodiment, different nanoparticles with different shapes can be used in case more than one target nucleic acid is to be detected.

Although silver and gold are the most commonly used materials for plasmonic nanoparticles theoretically any metal, alloy or semiconductor with a large negative real dielectric constant and small imaginary dielectric constant can be used. In one embodiment the nanoparticles are alloys of noble metal particles or mixtures of materials referred to herein. Other materials, such as aluminium, potentially offer advantages in refractive index sensitivity, different surface chemistries, and resonances into the ultraviolet, where many organic molecules absorb light. Copper can also be used as possible metal for the metallic nanoparticles.

In one embodiment, the metallic nanoparticles each have a size in at least one dimension or at least 10 nm or 20 nm or in a range of between about 10 nm to about 900 nm or in a range of between about 10 to 50 nm. In another example the size of the nanoparticles is in range of between about 10 to 30 nm, or 20 to 30 nm.

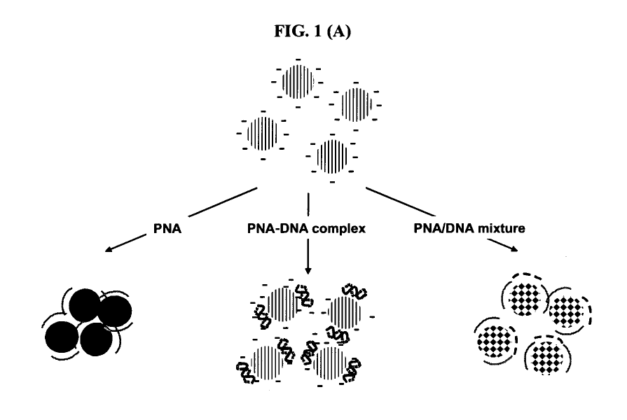

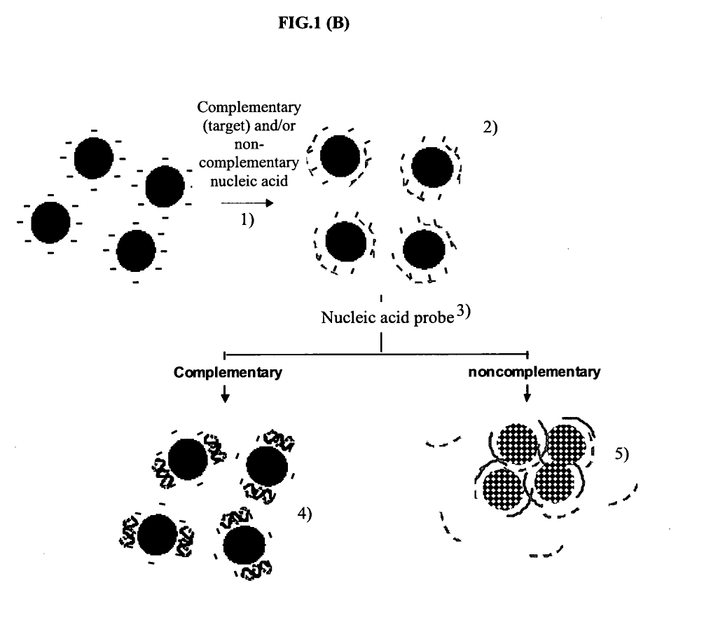

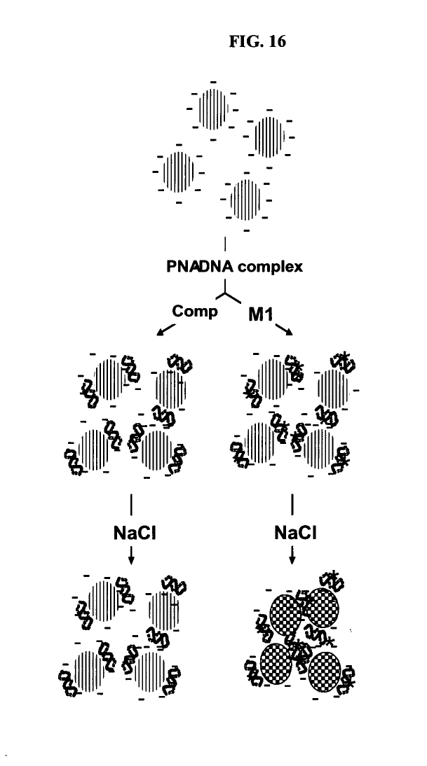

To avoid that the metallic nanoparticles aggregate even without that any nucleic acid is present negatively charged metallic nanoparticles are used. In one embodiment the negatively charged metallic nanoparticles are metallic nanoparticles carrying a negative charge at nanoparticle surface. Binding of a nucleic acid changes the repulsion of the metallic nanoparticles and thus leads to an aggregation. As more of the negative charges of the metallic nanoparticle are shielded by a nucleic acid as higher the degree of aggregation of the metallic nanoparticles. For example, binding of the nucleic acid probe with the substantially neutral net charge alone (i.e. target nucleic acid not present) leads to a high degree of aggregation (see also FIG. 1). It was found that binding of a hybrid of nucleic acids formed by complementary binding of the nucleic acid probe with its target nucleic acid does not aggregate the metallic particles to the same extent as binding of the nucleic acid probe with the substantially neutral net charge, due to the introduction of additional negative charges (the phosphate groups of the DNA or RNA backbone) to the particles. The different degree of aggregation can be measured. Furthermore, the degree of aggregation is also different in case the sample comprises a nucleic acid which is not the target nucleic and thus does not bind to the nucleic acid probe as illustrated in FIG. 1, right pathway. As illustrated in FIG. 16 and the corresponding examples referred to herein, the method of the present invention is sensitive enough to differentiate between single base mismatches. That means that the measurable degree of aggregation of the metallic nanoparticles changes if the target nucleic acid is not fully complementary to the nucleic acid probe.

Metallic nanoparticles with a negative surface charge can be nanoparticles wherein the negative charge of the metallic nanoparticles is conferred by a carboxylic acid or sulfonic acid or carbolic acid or a mixture of the aforementioned acids which is immobilized at the surface of the metallic nanoparticles.

In one embodiment, the carboxylic acid can be, but is not limited to citric acid, lactic acid, acetic acid, formic acid, oxalic acid, uric acid, pyrenedodecanoic acid, mercaptosuccinic acid, aspartic acid, to name only a few. Methods to immobilize such acids at the surface of metallic nanoparticles are known in the art. For example, citrate-stabilized AuNPs were prepared by thermal reduction of HAuCl₄ with sodium citrate (Grabar, K. C., Freeman, R. G., et al., 1995, supra). In brief, 500 mL of 1 mM HAuAl₄ was brought to a rolling boil with vigorous stirring. After that 50 mL of 38.8 mM sodium citrate was rapidly added, resulting in burgundy color. Boiling was continued for 10 min; the heating mantle was then removed, and stirring was continued for additional 15 min. Maya, L., et al. (2002, Langmuir, vol. 18, pp. 2392) discloses a method for immobilizing mercaptosuccinic acid at the surface of metallic nanoparticles. Tan, Y. N., et al., (2008, J. Phys. Chem. C, vol. 112, no. 14, pp. 5463) discloses a method for immobilizing aspartic acid at the surface of metallic nanoparticles. It was also found that the lower the surface charge density the higher for example the DNA or RNA detection sensitivity.

The nucleic acid probe can be fully complementary to the target nucleic acid or only partly complementary to the target nucleic acid. In other words, the nucleic acid probe can have a sequence comprising one or more non-complementary nucleotides. This allows detecting the presence and absence of a target nucleic acid comprising one or more mutations in its sequence compared to the sequence of the nucleic acid probe. As demonstrated herein and illustrated in FIG. 16, the method of the present invention can be used to differentiate between target nucleic acids which are fully complementary and which comprise, for example, one or more non-complementary nucleotide(s) in their sequence compared to the sequence of the nucleic acid probe.

To increase the sensitivity for the method of the present invention, for example to differentiate between target nucleic acids which are fully complementary to the sequence of the nucleic acid probe and which comprise one or more mutations in their sequence, it is possible to add a compound comprising cations, such as monovalent or divalent cations or mixtures thereof. Cations are used to screen the negative charges at the surface of the metallic nanoparticles and thus to induce more intensive aggregation.

Suitable salts that can be used herein include any inorganic salt. Any salt could increase the ionic strength of the mixture, screening the charge repulsion between the particles and between particles and the nucleic acid strand of the target nucleic acid. Examples, of inorganic salts include, but are not limited to NaCl, KCl, CaCl₂, BaCl₂, MgCl₂, NaBr, KBr, NaI, KBr, NaNO₃, KNO₃, Mg(NO₃)₂, Ca(NO₃)₂, Na₂SO₄, K₂SO₄, NaClO₄, NH₄Cl, NH₄NO₃, (NH₄)₂SO₄, CH₃COONa, CH₃COONH₄, to name only a few. The concentration of the cations in the solution can be in the range of between about 0.01 M to about 1 M, or between about 0.01 M to about 0.5 M, or between about 0.01 M to about 0.1 M.

To increase the sensitivity of the method referred to herein it is also possible to increase the temperature, for example, to increase the sensitivity for detecting single base mismatches. For example, it was found that a single base mismatch lowers the melting temperature (the stability) for about 13° C. It would therefore be possible to set the temperature in between the fully match and mismatch so as to enhance the sensitivity or selectivity for single base mismatch.

As previously mentioned, determining the presence, the absence or the amount of the target nucleic acid can be achieved by measuring the optical properties of the metallic nanoparticles or by measuring the size of the metallic nanoparticle aggregates formed.

The optical properties such as the absorbance can be determined with methods and devices known in the art, such as with a spectrophotometer. If the change of the optical properties is visible in the light wave range visible to humans it is also possible to determine the differences with the naked eye. The size of the nanoparticle aggregates which form upon binding of a nucleic acid can be determined with transmission electron microscopy (TEM), or light scattering techniques or a coulter counter for small particles.

The components used in the method of the present invention are preferably comprised in a solution, such as an aqueous solution. In one embodiment, the pH in the solution in which the method of the present invention is carried out is neutral (i.e. about pH 7). A neutral pH is used to avoid dehybridization of the hybrid complexes formed between nucleic acid probe and target nucleic acid. The temperature in the solution is preferably lower than the melting temperature of a hybrid complex formed between the nucleic acid probe and the target nucleic acid. All kinds of buffers can be used to conduct the method of the present invention therein, such as a phosphate buffered saline (PBS) or tris(hydroxymethyl)aminomethane (TRIS) buffer, to name only a few. In one embodiment, a buffer concentration in a range of between about 0.001 mM to about 500 mM can be used in the method referred to herein.

In a second aspect the present invention refers to a method of detecting presence or absence of a target nucleic acid. This method comprises:

incubate negatively charged metallic nanoparticles with a sample which is suspected to comprise the target nucleic acid to form a first mixture;

contacting the first mixture with a nucleic acid probe with a substantially neutral net charge to result in a second mixture; wherein the nucleic acid probe is substantially complementary to the target nucleic acid;

determining whether the target nucleic acid is comprised in the second mixture or not.

In a further aspect, the present invention refers to a kit for detecting presence or absence or for determining the amount of one or more target nucleic acid(s) in a sample. The kit can comprise at least one nucleic acid probe with a substantially neutral net charge described herein and/or at least one type of negatively charged metallic nanoparticle described herein.

In a further aspect the present invention is directed to at least one type of negatively charged metallic nanoparticle which can be used in a method or the kit of the present invention. Furthermore, the present invention is also directed to at least one nucleic acid probe with a substantially neutral net charge as described herein which can be used in a kit or a method of the present invention.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

Reagents

HAuCl₄.3H₂O (99.99%) and AgNO₃ (99.9%) were obtained from Alfa Aesar (MA). Trisodium citrate dihydrate (99.9%) was obtained from Aldrich and NaBH₄ from Fluka. Concentrated HCl and HNO₃ were all analytical grade and used without further purification. Mixed-base PNA oligomers of 10-, 13-, 20-, and 22-mer (denoted as PNA₁₀, PNA₁₃, PNA₂₀, and PNA₂₂) with no modification at C- and N-terminals, were synthesized by the Eurogentec S.A. Leige (Belgium). Target DNA of fully complementary (DNA_{comp}), noncomplementary (DNA_{nc}), and single-basemismatch sequence (DNAₘ₁) were from Proligo-Sigma (for PNA and DNA sequences, see Table 1).

TABLE 1
PNA and DNA Sequences

| Length | Name | Sequences | |
|---|---|---|---|
| 10-mer | PNA₁₀ | N′-ATCTTCTAGT-C′ | (SEQ ID NO: 1) |
| 13-mer | PNA₁₃ | N′-TTCCCCTTCCCAA-C′ | (SEQ ID NO: 2) |
| | DNA_{comp} | 5′-TTGGGAAGGGGAA-3′ | (SEQ ID NO: 3) |
| | DNAₘ₁ | 5′-TTGGGAGGGGGAA-3′ | (SEQ ID NO: 4) |
| 20-mer | PNA₂₀ | N′- TTGCACTGTACTCCTCTTGA-C′ | (SEQ ID NO: 5) |
| | DNA_{comp} | 5′-TCAAGAGGAGTACAGTGCAA-3′ | (SEQ ID NO: 6) |
| 22-mer | PNA₂₂ | N′-AACCACACAACCTACTACCTCA-C′ | (SEQ ID NO: 7) |
| | DNA_{comp} | 5′-TGAGGTAGTAGGTTGTGTGGTT-3′ | (SEQ ID NO: 8) |
| | DNA_{nc} | 5′-CAAAACAAAGATCTACATGGAT-3′ | (SEQ ID NO: 9) |
| | DNAₘ₁ | 5′-TGAGGTAGTAAGTTGTGTGGTT-3′ | (SEQ ID NO: 10) |

Colloidal Preparation

Citrate-stabilized AuNPs were prepared by thermal reduction of HAuCl₄ with sodium citrate (Grabar, K. C., Freeman, R. G., et al., 1995, Anal. Chem., vol. 67, pp. 735). Citrate-stabilized AgNPs were synthesized by the reduction of silver nitrate using sodium borohydride. Spherical AuNPs of 13.2±1.1 nm and AgNPs of 16.1±0.6 nm were observed under TEM. The concentrations of the AuNPs and AgNPs were 7.3 and 0.39 nM, respectively, calculated according to Beer's law, using the extinction coefficient of 2.7×10⁸ M⁻1 cm⁻1 for 13 nm AuNPs³⁰ and 9.4×10⁹ M⁻1 cm⁻1 for 16 nm AgNPs.

Characterization

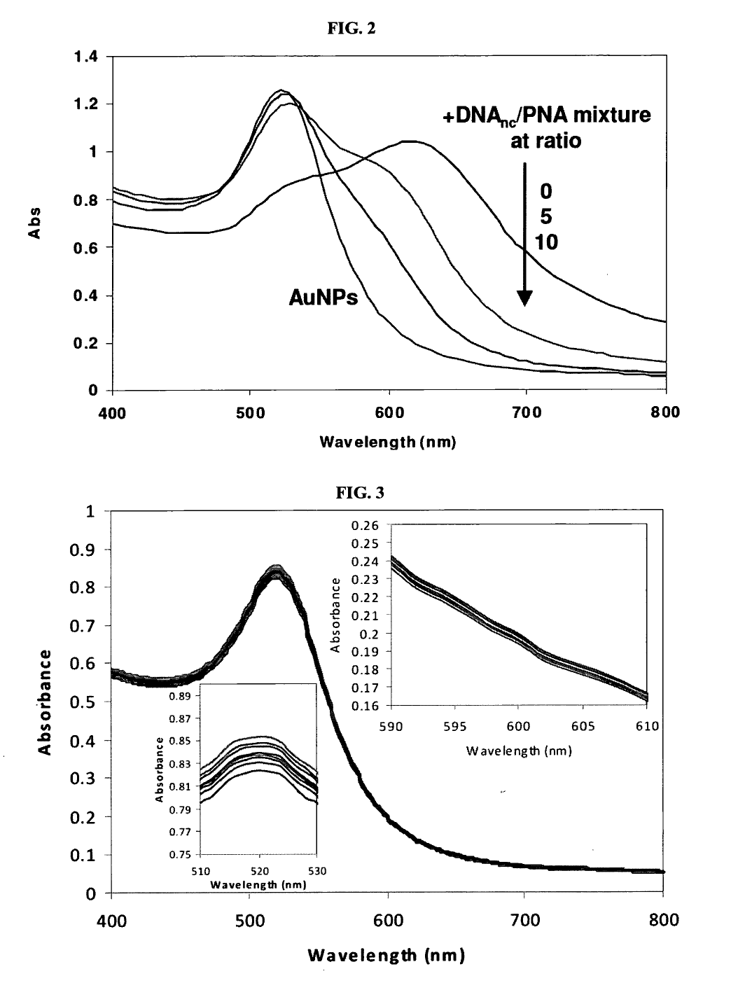

Ninety-six-well microplates were used as reaction carrier. UV-vis absorption characterization was performed using a TECAN infinite M200 (Tecan Trading AG, Switzerland). The reproducibility of the UV-vis absorption spectrum measurement (at room temperature) was evaluated through multiple scans of a given AuNPs (and AgNPs) solution aliquot in different wells. The variation of the absorbance measurement at representative wavelengths is about 1% for AuNPs and within 2% for Ag—NPs (FIGS. 2 and 3 and Table 2). ζ-potential measurements were preformed on bare AuNPs and PNA-, PNA-DNA complex-, ssDNA-, and dsDNA-coated AuNPs in water or in 0.15 M NaCl, using a ZETA PLUS zeta potential analyzer (Brookhaven Instruments). A mixture of AuNPs (7.3 nM) and nucleic acid samples (1 μM) of 450 μL was diluted into 2 mL for the measurement.

Assay Procedure

To test the coagulating property of PNA, aliquots of stock PNA probes (100 μM) were added to 150 μL of AuNPs (or AgNPs) solution. After 10 min incubation at room temperature, the color of the solutions was recorded using a camera and the absorption spectra were recorded in a wavelength range of 400-800 nm for AuNPs and 300-800 nm for AgNPs. To measure the ability of PNA-DNA complex and dsDNA to protect AuNPs (or AgNPs) and to detect the sequence specific hybridization, the PNA (or DNA) probe and DNA target of equal amount (40 μM) were annealed in 20 mM phosphate buffer (pH 7.2, containing 100 mM NaCl, and 0.1 mM EDTA), prior to addition to nanoparticle solutions (150 μL) (final DNA concentration 1 μM). When necessary, NaCl (final concentration 0.1-0.4 M) was added to accelerate particle aggregation. To test PNA hybridization with DNA inside AuNPs solution, DNA was first mixed with AuNPs (final DNA concentration 1 μM, final buffer concentration is 1 mM PBS). After 10 min incubation, PNA (final concentration 1 μM) was added.

PNA Binds More Strongly to AuNPs than its ssDNA Counterpart

FIG. 2 shows the stability of AuNPs solutions exposed to mixtures of the 20 mer PNA₂₀ (1 μM) and ssDNA of same sequence (no hybridization occurs) at ssDNA/PNA ratio of 0, 5, and 10. PNA-induced particle aggregation remains detectable when ssDNA is in great excess.

Reproducibility of UV-vis Spectrum Measurement for AuNPs and AgNPs

The reproducibility of the UV-vis spectrum measurement by performing multiple scans to a AuNPs solution (or a AgNPs solution) aliquot in eight different wells was evaluated. The obtained spectra from the eight wells overlapped extremely well (FIG. 2 and FIG. 3). The reproducibility, measured as relative standard deviations (RSD %) of the absorbance values at representative wavelengths (520 nm and 600 nm for AuNPs, 400 nm and 650 nm, as well as A650/A400 for AgNPs), is calculation (Table 2).

TABLE 2
Reproducibility of absorbance measurement

| | Absorbance at specific | | |
|---|---|---|---|
| Material | wavelengths (nm) | Average ± SD | RSD % |
| AuNPs | A520 | 0.8391 ± 0.009665 | 1.15 |
| | A600 | 0.1970 ± 0.002022 | 1.03 |
| AgNPs | A400 | 1.3441 ± 0.2450 | 1.82 |
| | A650 | 0.06436 ± 0.001014 | 1.58 |
| | A650/A400 | 0.05020 ± 0.0004855 | 0.97 |

Surface Plasmon Resonance (SPR) Detection of DNA Hybridization with Immobilized PNA₁₃ Probe

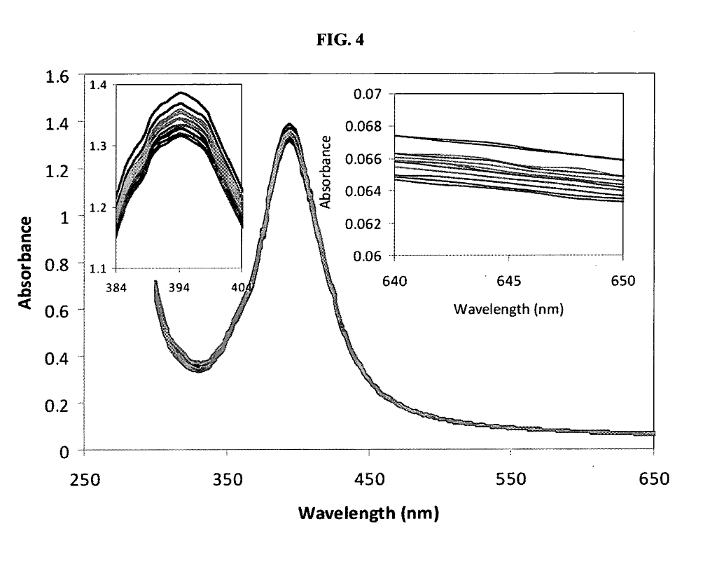

A biotinylated PNA₁₃ is immobilized on SPR gold disk using streptavidin-biotin interaction. The hybridization of fully complementary and single-base-mismatched target DNA (DNA_{comp} and DNAₘ₁) were monitored in real-time (FIG. 4). Under tested buffer solutions, 1 mM PBS and 0.1 mM PBS, both targets hybridize equally well and no mismatch discrimination was obtainable.

PNA-Induced AuNPs Aggregation

A study of PNA-induced AuNPs aggregation with the involvement of four mixed-base PNA oligomers of different chain length (10-, 13-, 20-, and 22-mer) and base composition was conducted, aiming to extract PNA-AuNPs binding characteristics in terms of concentration, sequence, and chain length dependence.

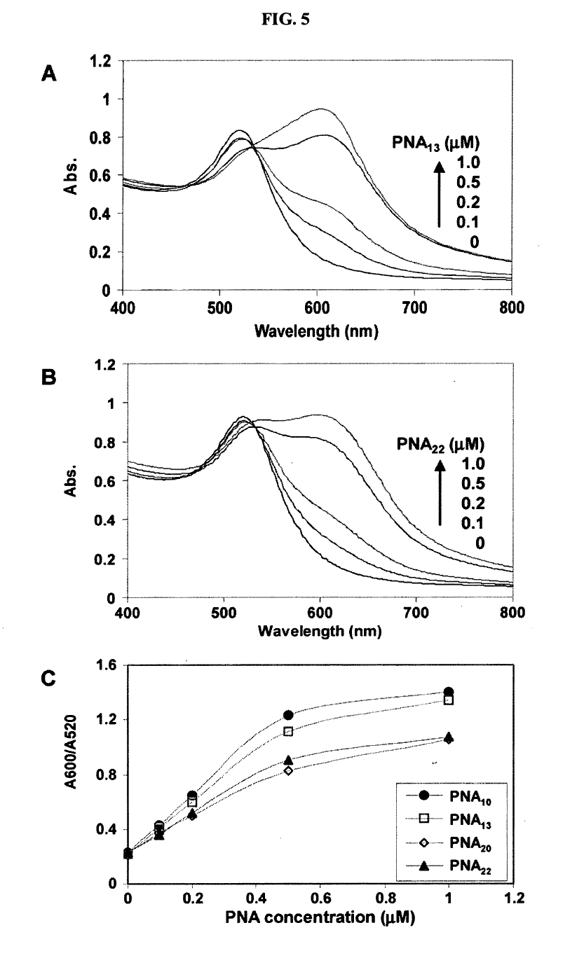

Parts A and B of FIG. 5 are representative UV-vis adsorption spectra of AuNPs mixed with the 13-mer and 22-mer PNA (PNA₁₃ and PNA₂₂) at different concentrations. The shift of surface plasmon peak from 520 nm to longer wavelengths evidences the particle aggregation. The degree of aggregation is found to be determined by PNA concentration. The plots of aggregation degree (measured as ratio of absorbance at 600 and 520 nm, A600/A520) versus PNA concentration for all four PNAs (FIG. 5C) show that the shorter PNA₁₀ and PNA₁₃ aggregate the particles more effectively than the longer ones (PNA₂₀ and PNA₂₂) at any given concentration. This implies that the shorter PNAs cover the AuNPs more effectively than the longer ones. This characteristic resembles what is known for ssDNA, which has been rationalized as the shorter ones having a less coiled structure that ensures the nucleobases to be exposed more easily. As for the sequence effect, the similar aggregation efficiency of PNA of similar length but different base composition (10- and 13-mer; 20- and 22-mer) shows that the multivalent interaction between mixed-base PNA and AuNPs is not sensitive to the base sequence.

To better understand the mechanism of PNA induced AuNPs aggregation, the surface charge of AuNPs before and after PNA coating (PNA₁₃ was used as an example) was measured. It was found that PNA₁₃-coated AuNPs has a similar charge density (ζ-potential −20.11±3.6 mV) as compared to the uncoated AuNPs (ζ-potential −21.04±2.6 mV). This suggests that the PNA coating only shields the citrate ions (no displacement occurs). This concurs with previous Raman spectroscopy result for single-stranded oligonucleotides (ssDNA), showing that coating of ssDNA on AuNPs did not displace citrate ions. However, the inability of charge neutral PNA oligomers to displace citrate ions from gold surface appears to conflict with previous studies for other charge neutral substances, e.g., adenosine, nucleobases, and nucleosides.

Coating of these small molecules has been proven to be able to displace weakly bound citrate ions from AuNPs. It might be the steric hindrance arising from the polymer structure of the PNA oligomers makes the binding not strong enough to displace the citrate ions. Using DNA as an example, it was previously shown that ssDNA oligonucleotides bind less effectively to AuNPs than mononucleosides due to larger molecular size.

PNA Binds More Strongly to AuNPs than its ssDNA Counterpart

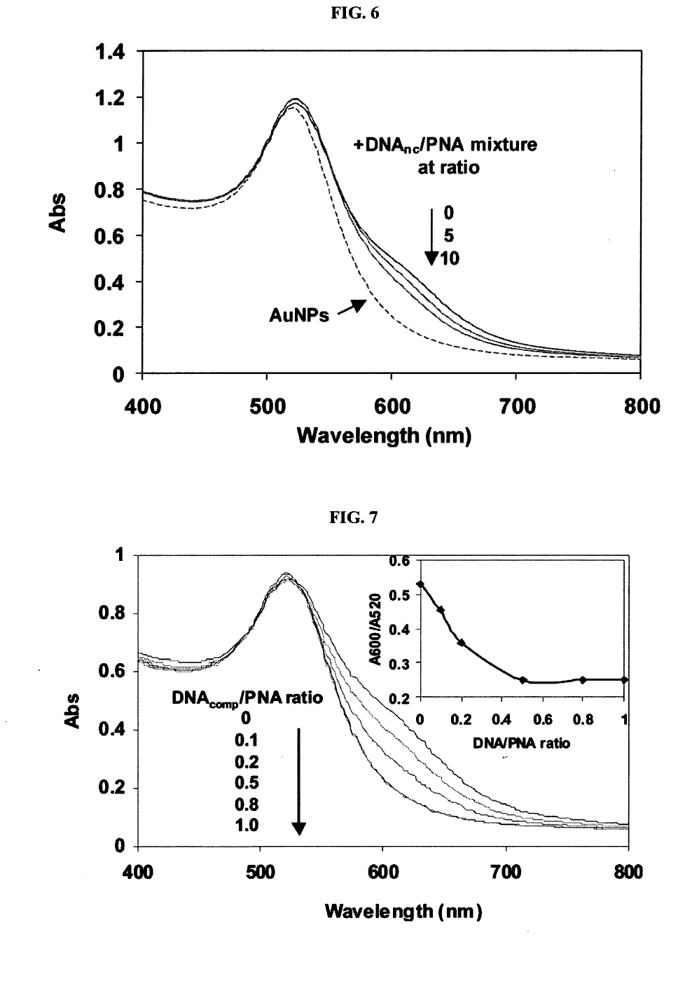

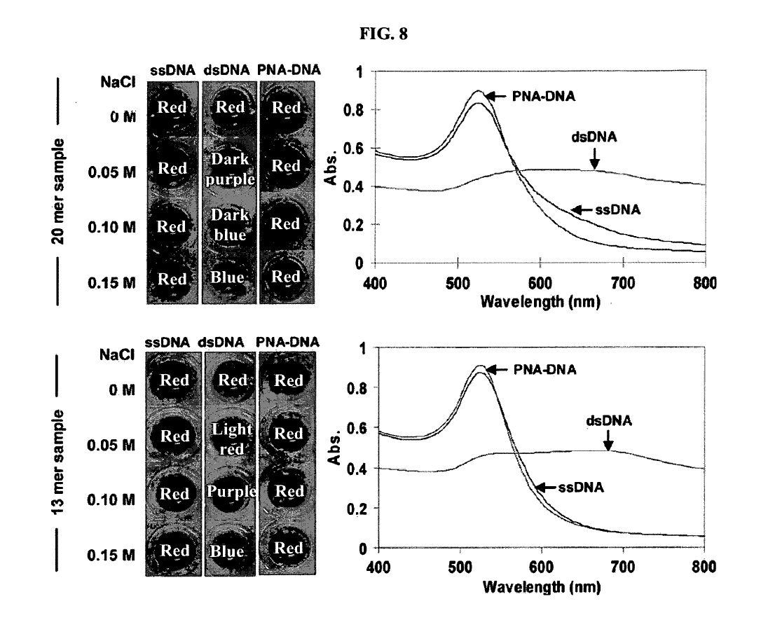

ssDNA is known to be able to adsorb on AuNPs and to stabilize colloidal suspensions in high salt concentration. However, in the presence of PNA, it was found that ssDNA's stabilization effect is abolished. FIG. 6 shows the stability of AuNPs solutions exposed to mixtures of PNA₂₂ (200 nM) and ssDNA of the same sequence (no hybridization occurs) at a ssDNA/PNA₁₃ ratio of 0, 5, and 10. FIG. 2 shows the result with the 20-mer PNA₂₀ at a higher concentration (1 μM) and same range of ssDNA/PNA ratio. Both FIG. 6 and FIG. 2 show that PNA-induced particle aggregation remains largely detectable when ssDNA is in great excess. When NaCl is added, further aggregation was observed regardless the presence of ssDNA (ssDNA alone at the above tested concentrations is sufficient to protect AuNPs to against salt induced aggregation; see FIG. 8 and related discussion). The abolishment of ssDNA's protection effect when PNA is present gives strong evidence that PNA binds dominantly to AuNPs relative to ssDNA. It is believed that the distinct backbone properties of PNA (neutrality, high rigidity, and peptide composition) are attributable to its higher affinity to gold. The neutrality ensures no charge repulsion between PNA and citrate coated AuNPs as severely encountered by negatively charged ssDNA. The high backbone rigidity renders the nucleobases to be exposed more effectively than those in coiled ssDNA. The peptide composition introduces secondary interaction with gold.

Stabilization Effect of PNA-DNA Complex

Apart from the fact that non-complementary DNA (denoted as DNA_{nc}) has little interference to PNA-induced AuNPs aggregation, further experiments with complementary DNA_{comp} show that the presence of a small amount of DNA_{comp} can disrupt the particle aggregation significantly. FIG. 7 shows the discriminative stability of AuNPs solutions mixed with PNA₂₂ (200 nM) and PNA₂₂ annealed with its fully complementary target DNA_{comp} at DNA/PNA ratio of 0.1 to 1. The PNA-induced particle aggregation is depleted gradually with the increase of DNA_{comp} concentration until being abolished at DNA/PNA ratio of 0.5.

Apparently, the depletion of particle aggregation in the presence of DNA_{comp} is due to the reduction of free PNA upon formation of the PNA-DNA complex. The observation that particle aggregation is abolished when half of the PNA (at DNA/PNA ratio of 0.5) being consumed is evidence that the as-formed PNA-DNA complex prevents the remaining PNA (100 nM in this case) from aggregating the particles (PNA₂₂ at 100 nM is sufficient to induce detectable aggregation in the absence of other species, FIG. 5B). This is a primary indication that PNA-DNA complex has certain affinity to AuNPs. The binding of the PNA-DNA complex provides sufficient protection to AuNPs to overcome PNA's destabilization effects.

To further understand the binding property of PNA-DNA complex to AuNPs and to compare its protection of AuNPs relative to ssDNA and dsDNA, PNA₁₃ and PNA₂₀ and their corresponding complementary DNA of equal amount were annealed to form PNA-DNA complexes. The incubation solutions were then mixed with AuNPs. The stability of the colloidal solutions before and after addition of salt was recorded (FIG. 8). Before addition of NaCl (0 M), the solutions with ss-DNA, dsDNA, and PNA-DNA complex are all stable as indicated by their red color and sharp SP peaks around 520 nm. Slight peak wavelength and peak intensity difference are a primary indication of the adsorption of different nucleic acid samples to gold that leads to the local change of the dielectric permittivity (spectra before adding salt not shown). After NaCl is added, the dsDNA containing solutions gradually aggregated as expected. With the increase of NaCl concentration, the color changes from red to purple and blue. However, the other solutions containing either PNA-DNA complex or ssDNA remain in red color, even at the highest NaCl concentration tested. From the similar color code of these two types of solutions, one can not deduct a stability difference; whereas their UV-vis adsorption spectra (at 0.15 M NaCl) reveal a noticeable difference in particles' stability. For both the 13- and 20-mer samples, the ssDNA-containing solutions are less stable than the PNA-DNA containing solutions, showing a noticeable red-shift of the spectrum. It is well-known that duplex DNA (dsDNA) has little affinity to negatively charged AuNPs due to its stable DNA-DNA double-helix geometry that always isolates the nucleobases and presents the negatively charged phosphate backbone. As a result, dsDNA cannot protect AuNPs from salt-induced aggregation, as compared to ssDNA.

The discovery that despite the presence of double-helix geometry PNA-DNA duplexes can effectively protect AuNPs against salt induced aggregation, better than dsDNA and even ssDNA, is an interesting phenomenon. This phenomenon was rationalized from both the electrostatic and steric stands. First of all, a PNA-DNA duplex carries only half of the negative charges relative to its dsDNA counterpart. Under the low ionic strength conditions used in this experiment (<3 mM NaCl), the effective charge of PNA-DNA complex would be noticeably lower than that of dsDNA. This would enable the PNA-DNA complex to adsorb more effectively than dsDNA onto citrate ion-coated AuNPs, due to depleted charge repulsion. Second, the nitrogen-/oxygen-containing peptide backbone of the PNA strand can interact with gold, leading to a higher affinity than dsDNA. In a comparison between PNA-DNA complex and ssDNA, both having similar charge density but entirely different structure properties (insulated or exposed bases), it is not possible to speculate which one binds more effectively to gold with a higher affinity without characterizations using other techniques. But the stronger protection of PNA-DNA than ssDNA could be largely or at least partially attributable to the larger molecular size and the structure rigidity of PNA-DNA complex that introduces more steric protection than ssDNA. This interpretation is indirectly supported by the fact that the stability difference between PNA-DNA complex- and ssDNA-protected AuNPs is larger for the 20-mer samples than the 13-mer samples (more obvious UV-vis spectra difference in FIG. 8 “20 mer sample” than in “13 mer sample”). It would be expected that the steric effect of the larger PNA-DNA duplexes is more obvious than the smaller ones.

The hypothesis that steric effect, rather than charge effect, is attributable for the better stability of PNA-DNA complex-protected AuNPs than ssDNA protected ones has been further supported by ζ-potential measurement (using the 13-mer sample as example). In 0.15 M NaCl, the ζ-potentials of AuNPs coated with PNA-DNA, ssDNA, and dsDNA are −31.2±8.2, −30.7±6.7, and −10.4±7.3 mV, respectively. The charge density of the PNA-DNA complex- and ssDNA protected AuNPs are similar, meaning that electrostatic repulsion is similar. Thus, the observed differential stability should be a result of differential steric effect. For the dsDNA-protected particles, the high ζ-potential value, indicating of reduced surface charge and reduced electrostatic repulsion, explains why aggregation occurs easily.

Thus, it was demonstrated that (1) mixed-base PNA oligomers can induce immediate particle aggregation, (2) PNA oligomer have a higher affinity to NPs than its ssDNA counterpart, (3) PNA-DNA complex, although having a stable double helix structure similar to dsDNA, can effectively protect NPs from salt induced aggregation, and (4) the ability of different nucleic acids to protect AuNPs against salt-induced aggregation is in the order PNA-DNA complex>ssDNA>dsDNA.

Nucleic Acids' Binding Characteristics to AgNPs

AgNPs have a higher extinction coefficient relative to AuNPs of the same size. It is therefore believed that AgNPs would have a higher sensitivity for use in colorimetric assays. Therefore, experiments were conducted with citrate ion protected AgNPs to extend the understanding of nucleic acid-AgNPs interactions and to address the sensitivity merit of this material for this particular application.

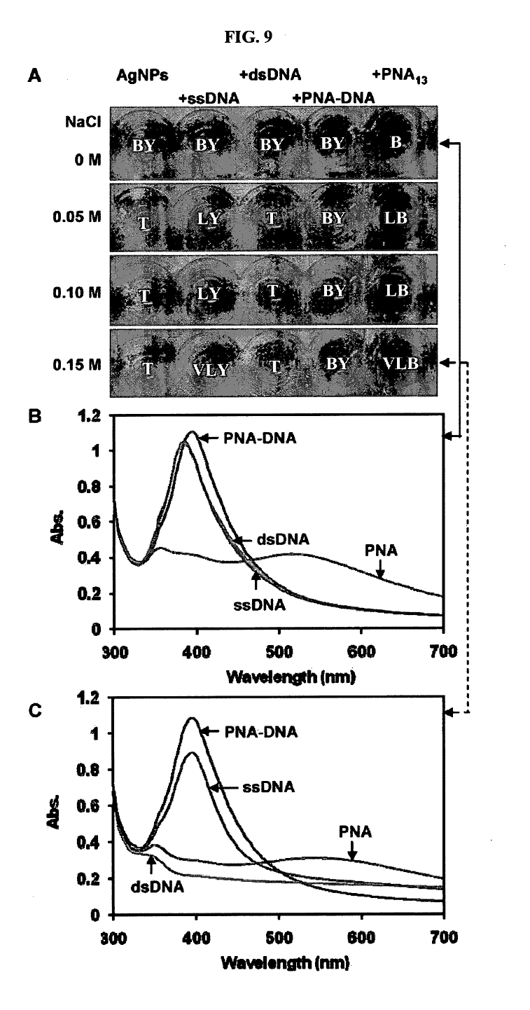

FIG. 9 shows the stability of AgNPs solutions incubated with ssDNA, dsDNA, PNA-DNA complex, and PNA (13-mer samples). From the color photographs and the UV-vis adsorption spectra, the following observations were made: (1) mixed-base PNA can induce immediate AgNPs aggregation, characterized as color change from yellow to brown and the drop of absorbance at the original SP peak (˜400 nm) and the appearance of SP peak at longer wavelength; (2) PNA-DNA hybridization disrupts the PNA-induced aggregation; and (3) PNA-DNA complex, ssDNA, and ds-DNA do not affect AgNPs' intrinsic stability, but when NaCl is added, dsDNA-containing AgNPs aggregates immediately at the lowest NaCl concentration tested, ssDNA-containing AgNPs aggregates slowly with the increase of salt concentration, and the PNA-DNA complex-containing AgNPs remains stable up to the highest salt concentration tested.

With these observations, we confirm that nucleic acids' binding characteristics and their stabilization effects are identical for AuNPs and AgNPs. The well-known characteristic that ssDNA, but not dsDNA, can protect AuNPs against salt induced aggregation is, for the first time, proven true for AgNPs. With this experiment it was also found that AgNPs are more sensitive in response to small difference in stabilization effect. The previous finding that PNA-DNA complex can better protect nanoparticles against salt-induced aggregation than ssDNA is further confirmed with Ag—NPs, via both the color change and UV-vis spectrum shift, whereas with AuNPs, only a slight spectrum shift is accountable for this characteristic (FIG. 8).

Detection of Pre-Annealed PNA-DNA Complex with Single-Base-Mismatch Sensitivity Using AuNPs and AgNPs

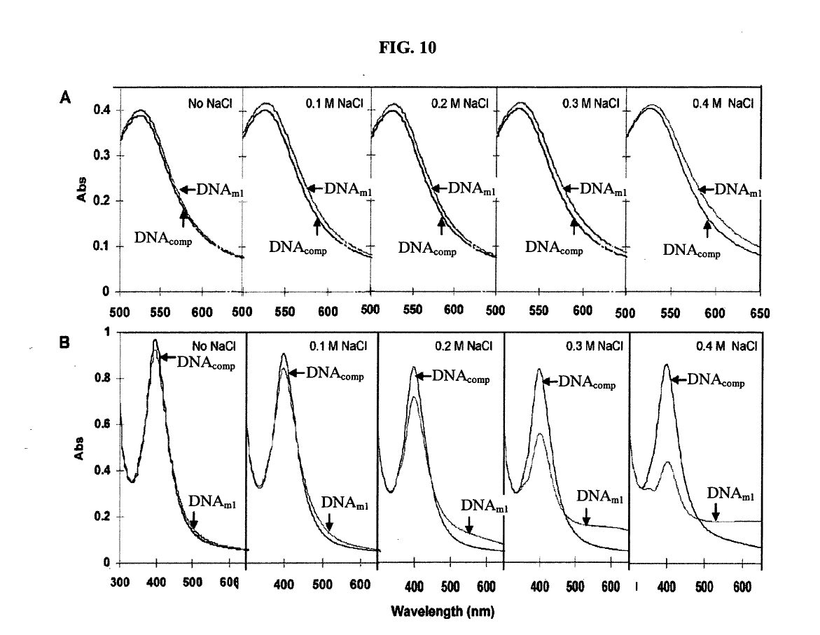

Successful hybridization of PNA with its complementary DNA_{comp} can abolish PNA-induced particle aggregation. In the case where target DNA contains a single-base-mismatch (DNAₘ₁), the less effective hybridization (or higher tendency of dehybridization) would cause some PNA to remain free in solution. It was thus anticipate that the unhybridized PNA would aggregate the particles, detectable by typical spectrum shifts. FIG. 10 shows the experimental validation using the 13-mer sample. In the absence of NaCl, the UV-vis curves of the NPs solutions containing PNA annealed with DNAₘ₁ carry a small but noticeable signature of particle aggregation, i.e., an increase of A600 of 3.5% for AuNPs and an increase of A600/A400 of 10.8% for AgNPs, relative to those with fully complementary DNA_{comp}. These spectra shifts are significant compared to the variation of A600 measurement for AuNPs (1.03%) and A600/A400 measurement for AgNPs (0.97%) (FIGS. 3, 4 and Table 2). When NaCl is added to enhance the stringency, the discrimination between DNA_{comp} and DNAₘ₁ is enlarged. For AuNPs, the ΔA600 value increases to 8.5%, 11.0%, 15.3%, and 21.1% at NaCl concentrations of 0.1, 0.2, 0.3, and 0.4 M, respectively; for AgNPs, the ΔA600/A400 increases to 23.9%, 108.7%, 291.6%, and 316.5% at the same NaCl concentration range. At a fixed salt condition, a larger degree of differentiation is observed for AgNPs, which proves again that AgNPs is more sensitive in response to small difference in stabilization effect and is a more sensitive colorimetric platform for single-mismatch detection.

Detection of PNA Hybridization with DNA in Pre-Incubated DNA/AuNPs Mixtures

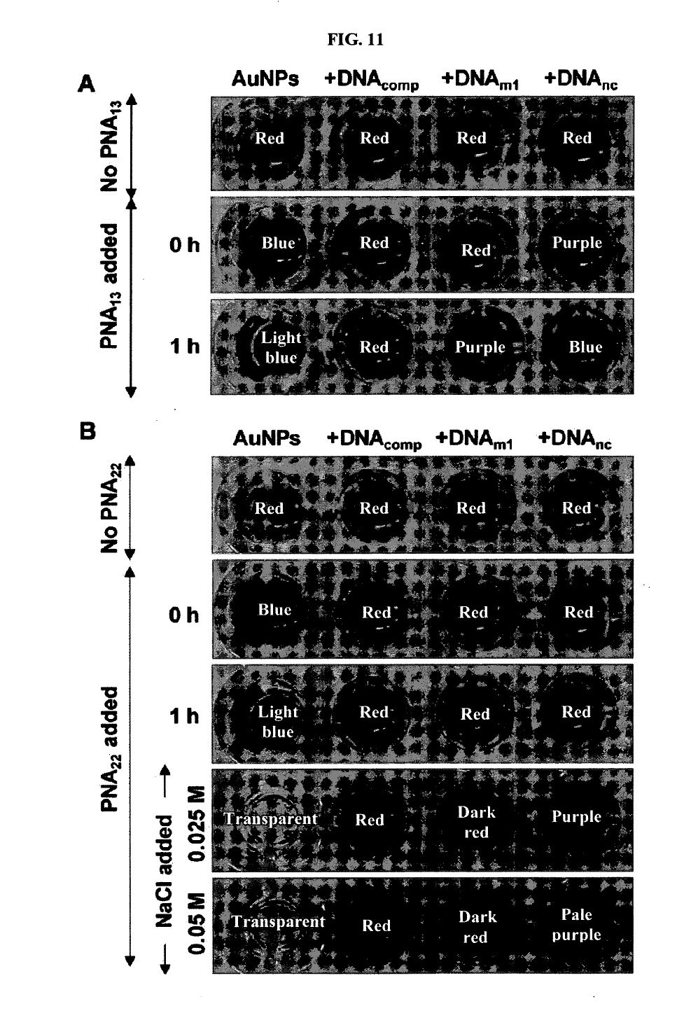

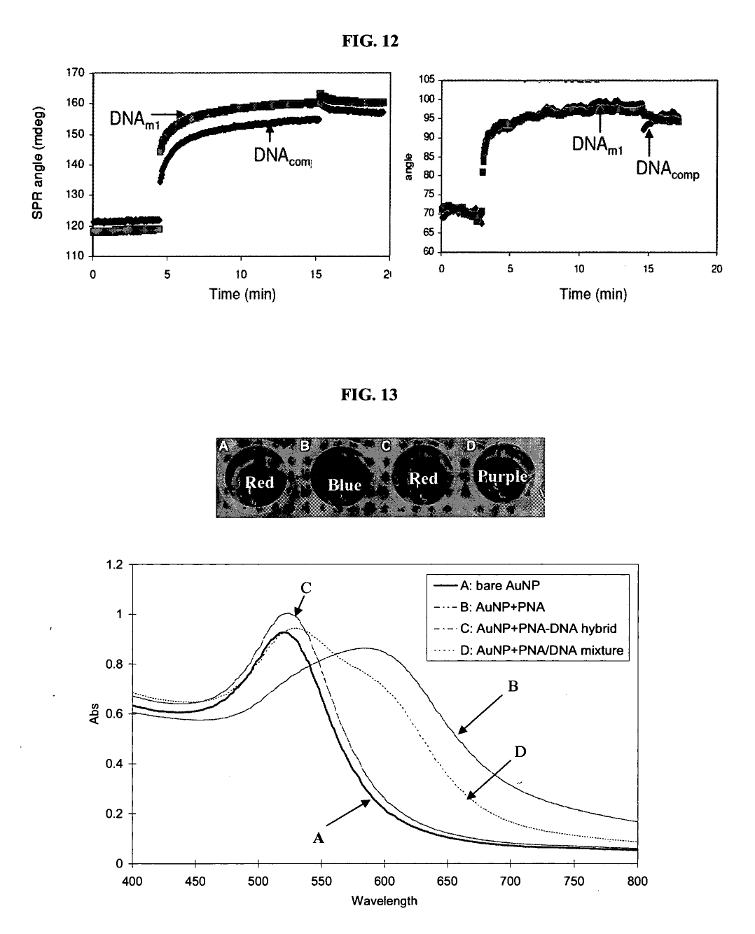

According to the characteristic that PNA has a stronger affinity to AuNPs than ssDNA, an alternative approach was developed to detect a specific DNA through PNA-DNA hybridization in a DNA/AuNPs mixture but not through post addition of pre-annealed PNA-DNA complex. This approach is designed on the basis of the indication that PNA is able to strip DNA from the AuNPs surface and further hybridize to the DNA if the sequences are complementary. FIG. 11A shows the results with the 13-mer samples. Prior to PNA addition, AuNPs solutions mixed with different target DNA (DNA_{comp}, DNAₘ₁, and DNA_{nc}) are all in red, showing a good dispersion state. When PNA₁₃ is added, gradual color changes are developed in the single-base-mismatch (DNAₘ₁) and non-complementary (DNA_{nc}) wells, but not in the complementary DNA_{comp} well. This confirms the assumption that PNA can displace adsorbed DNA and induces particle aggregation when it is free from hybridization (in the case of DNA_{nc}) or hybridizes in a lower efficiency (the case with DNAₘ₁), whereas when the sequences are complementary, displacement of DNA is accompanied by an effective PNA-DNA hybridization. The resulting PNA-DNA complexes keep the AuNPs stable. The same experiment was repeated with the PNA₂₂ and its target DNA (DNA_{comp}, DNAₘ₁, and DNA_{nc}) (FIG. 11B). Interestingly, it was found that with time passes (up to 2 h), no obvious color changes are developed in any of the wells upon PNA₂₂ addition, which means that PNA₂₂ failed to displace (and hybridize with) the adsorbed ssDNA from AuNPs surface or the displacement is insignificant. This discrepancy relative to the PNA₁₃ could be explained by the previous discovery that a longer PNA is less effective than a shorter one to coat on AuNPs. To further confirm our speculation that there might be a small degree of displacement of DNA by PNA₂₂ (too small to induce particle aggregation), NaCl was added to screen the negative charges. Upon a proper selection of salt concentration (0.025 and 0.05 M), obvious color discrimination is observed between wells containing DNA_{comp}, DNAₘ₁, and DNA., respectively. The color changes to dark red (for DNAₘ₁) and to purple/blue (for DNA.) are an indication of particle aggregation. The retained stability of AuNPs in the DNA_{comp} well with the exposure to NaCl must be originated from the formation of PNA-DNA complex which has a strong ability to protect AuNPs, as demonstrated using the pre-annealed PNA-DNA complex earlier. The successful discrimination of single-base mismatch using PNA- and PNA-DNA complex controlled AuNPs/AgNPs aggregation/dispersion demonstrates the advantageous of the colorimetric assay relative to the solid-liquid phase hybridization assay, e.g., using surface plasmon resonance spectroscopy (SPR). With the SPR measurement of DNA hybridization to immobilized PNA probes, no discrimination is detectable between DNA_{comp} and DNAₘ₁ for the 13-mer sample (FIG. 12) and for the 22-mer sample, unless stringent hybridization conditions are used.

### Further Examples I to V

In the following one-component assays and two-component assays are described for detecting nucleic acids. Depending on whether the AuNPs and AgNPs are used separately (Examples I to IV) or used in mixture (Example V), one-component assays and two-component assays have been used. Color change relies on either the change of nanoparticles' intrinsic stability (Examples I, IV, V) or the change of their stability against salt (Examples II, III-V).

### Example I

### Detection of the Presence of a Specific DNA Using AuNPs' Intrinsic Stability

As described above, neutrally charged PNA and negatively charged DNA (or PNA-DNA hybrids) exert different effects on AuNPs' stability and aggregation behavior. FIG. 13 shows the photographs and UV-vis spectra of AuNP solutions under exposures to different nucleotide samples. The citrate anions protected AuNPs is well dispersed, showing red color (A). The addition of a charge neutral PNA probe (20 mer) to the AuNPs solution (final concentration of PNA is 1 μM) caused an immediate particle aggregation observed as color changes to blue (B). It is believed the PNA probe interacts strongly with the gold nanoparticles, through coordination chemistry between nitrogen/oxygen containing nucleosides and gold, causing obscurity of the citrate anions thereby removes charge repulsion between the nanoparticles, which causes the nanoparticles to aggregate.

When a PNA-DNA hybrid solution (equal amount of PNA and its complementary DNA were annealed to form hybrids in 10 mM phosphate buffer pH 7.2, 100 mM NaCl, and 0.1 mM EDTA) was added, the AuNPs solution remained stable and no aggregation took place. Color of the solution remained red (C). This is because capping of AuNPs with PNA-DNA complex retains sufficient negative charges (from the phosphate backbone of the DNA strands) on the particle surface, even though citrate anions are shadowed in the process.

When an irrelevant DNA was mixed with PNA probe (no formation of PNA-DNA complex due to non-match in sequence), certain extent of aggregation in the solution takes place, and color changes to purple (D), showing that the un-hybridized PNA probe binds dominantly to the particles, resulting in aggregation due to obscurity of the negative charges.

From the corresponding UV-vis spectra, it can be seen that there is a red shift of surface plasmon (SP) peak (λₘₐₓ) from 520 nm for the well dispersed solution A and C to a longer wavelength of 600 nm for aggregated B and D. The spectra shift leads to an increase of absorbance at 600 nm and a decrease at 520 nm. The A600/A520 absorbance ratio value can be used as a quantitative measure of the aggregation extent. For solution C containing specific DNA hybridized to the PNA, the A600/A520 ratio is 0.26, whereas for solution D with a nonspecific DNA, the A600/A520 ratio is 0.76. Hence, increase of A600/A520 ratio between the two systems is 192%.

To determine the DNA detection sensitivity of the current colorimetric assay, different amount of target DNA (0-200 nM) was added to PNA probe (200 nM) solution for hybridization. A detection limit of <10 nM was obtained for the 13 mer DNA. The nanomolar sensitivity is comparable with results from other label free assays such as Surface Plasmon Resonance (SPR).

### Example II

### Detection of the Presence of a Specific DNA Using AuNPs' Stability Against Salt

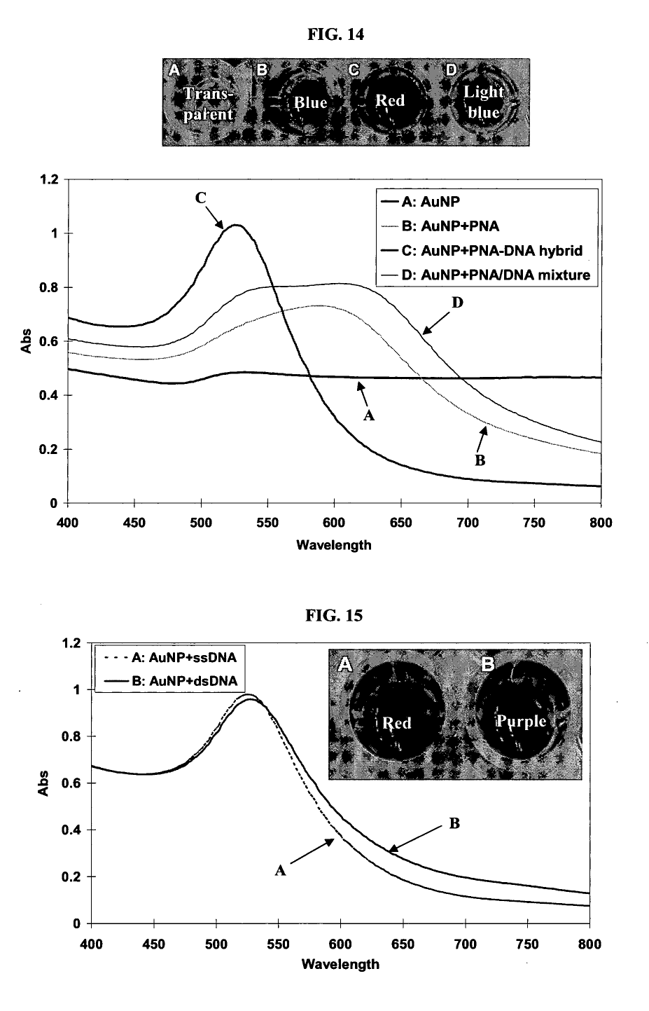

As discussed above, electrostatic repulsion between anions, such as citrate anions is essential to protect nanoparticles from aggregation. When this electrostatic repulsion is screened by salt (e.g. sodium chloride, NaCl), the nanoparticles tend to aggregate. NaCl is added into each solution from FIG. 13 (final concentration is 0.1 M) to test stability of these solutions against salt, by observing the color change and the shift of UV-vis curves (FIG. 14).

For bare AuNPs without addition of nucleic acids (A), 0.1 M NaCl caused the particles to precipitate from the solution and the solution became colorless (no UV-vis absorption). In the presence of PNA (B) and PNA/DNA mixture (D), addition of NaCl led to further aggregation shown by the blue color and the further shift of UV-vis curves to longer wavelengths. On the other hand, in the presence of PNA-DNA hybrid (C), the particles remain well dispersed under 0.1 m NaCl as shown by the red color and the SP peak at 520 nm. Thus, test of AuNPs' stability against salt further affirms that presence of a specific target DNA which forms PNA-DNA hybrid can protect AuNPs from salt induced aggregation. To quantify the aggregation extent, the A600/A520 ratio was calculated for solution C (containing specific DNA hybridized with the PNA) and solution D (containing a non-specific DNA). The value was 0.32 and 1.069 respectively. The increase of A600/A520 ratio between the two systems is 243%, being slightly higher than that (192%) using intrinsic stability of nanoparticles alone.

Use of PNA as probe for colorimetric detection of DNA is more sensitive than that used in prior-art, which uses differential electrostatic interaction of single-stranded DNA (ssDNA) and double strand (dsDNA) with AuNP as colorimetric sensing principle. FIG. 15 shows the results obtained using the prior-art method, i.e. photograph and UV-vis spectra of ssDNA and dsDNA treated AuNP solutions after addition of 0.1 M NaCl. Successful hybridization forming dsDNA caused an obvious AuNP aggregation, because the strand rigidity and doubled negative charge of dsDNA affects ability of dsDNA to coat on AuNP, thus causing a lower stability against salt. A600/A520 ratio value shifted from 0.29 for the unhybridized solution (ssDNA) to 0.54 for the hybridized solution. Increase of A600/A520 ratio is merely 54%, being much smaller than that of the current method (245%) using PNA as probe.

### Example III

### Detection of Single-Base-Mismatch Using AuNPs' Stability Against Salt

Detection of single-base-mismatch in DNA is important for diagnosis of generic disease. The detection method of the present invention can also be used for this purpose through use of AuNP's stability against salt, as shown in the scheme in FIG. 16.

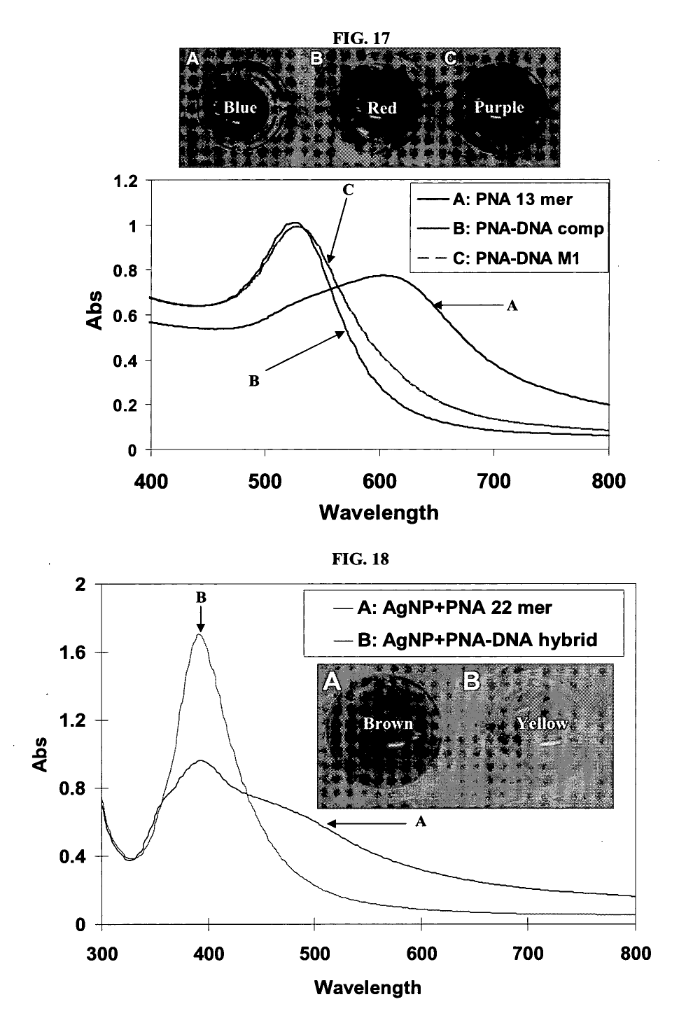

PNA-DNA duplexes of fully complementary sequence and single-base-mismatch sequence have undistinguishable effect on AuNPs' intrinsic stability. Discrimination relies on a high stringent condition, with for example, an additional of NaCl (FIG. 17). A very obvious difference between fully complementary DNA (B) and single-base-mismatch DNA (C) is observed both from the color difference and the UV-vis spectra shift in the presence of 0.1 M NaCl.

It is believed that due to the presence of the mismatched base, the PNA-DNA hybridization efficiency is reduced, leaving some PNA unhybridized. The free PNA in the solution would bind in competition to AuNPs with negatively charged PNA-DNA hybrids and the ssDNA, and to deplete the stability of the nanoparticles, causing certain degree of aggregation. To quantify the particle aggregation in the presence of SBM target, the A600/A520 was calculated for both the complementary (0.44) and mismatch DNA (0.29). The value increases by 51.6%.

The same characteristic was obtained for a longer sequence of 22 mer. Detection of single-base-mismatch in a longer sequence (22 mer in this case) is more difficult than in a shorter sequence (13 mer sequence). To quantify particle aggregation in the presence of SBM for the 22mer target, the A600/A520 was calculated for both the complementary (0.43) and mismatch DNA (0.36). The value increases by 17.6%, being smaller than that (51.6%) for the mismatch detection in the 13 mer sequence. Successful detection of single-base-mismatch in the 22 mer sequence at room temperature is of significance. Other solid-liquid phase biosensor methods (SPR and electrochemical methods) can not detect single-base-mismatch in this sequence at room temperature without attempts to increase the hybridization stringency, such as using high temperature.

### Example IV

### Detection of Specific DNA Sequence and Single-Base-Mismatch Using AgNPs

Silver nanoparticles (AgNPs) have also been used for a colorimetric assay. Citrate anion coated AgNPs solution is well dispersed, showing a yellow color (the SP peak is around 400 nm). Addition of a 22 mer PNA caused the AgNPs to aggregate, showing color and UV-vis spectrum changes (FIG. 18). The remarkable decrease in the absorbance at the SP peak wavelength (400 nm) and the red shift of the spectra (i.e. increased absorbance at the longer wavelength, e.g. 550 nm) are typical signatures of AgNPs' aggregation. A550/A400 ratio can be used to provide quantification of the aggregation extent and hence the target DNA amount. In contrast, the PNA-DNA hybrid protected AgNP remain in yellow color with a corresponding sharp SP peak at 400 nm, showing that the particles are stable and well dispersed under the protection of negatively charged PNA-DNA complex.

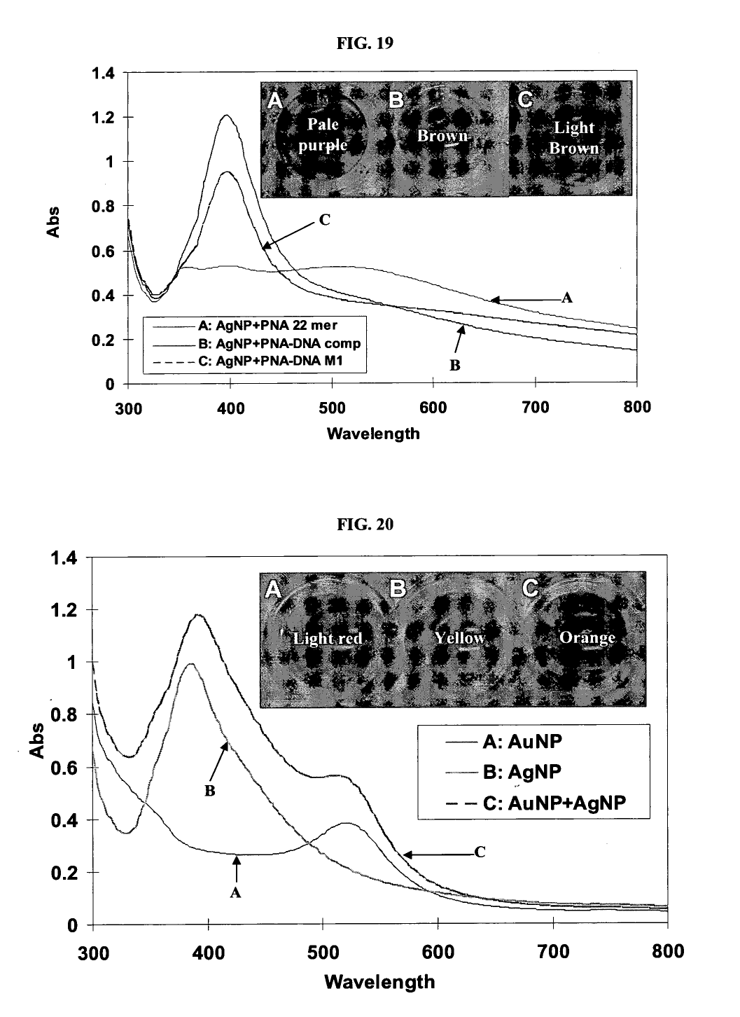

Single-base-mismatch detection using AgNP's stability against salt is also demonstrated (FIG. 19). An obvious decrease in A400 and spectrum shift to longer wavelengths were observed for the target DNA containing a single-base-mismatch.

When AuNPs and AgNPs are used in mixture, a two-color-change assay is used. FIG. 20 shows the original color of AuNPs, AgNPs, and a mixture of the two types of particles. As expected, the spectrum of the mixture is simply a summation of the two individual spectra for the silver and gold nanoparticles, showing two peaks at 400 nm and 520 nm. These two peaks will be referred for parallel detection of DNA to eliminate potential experimental errors.

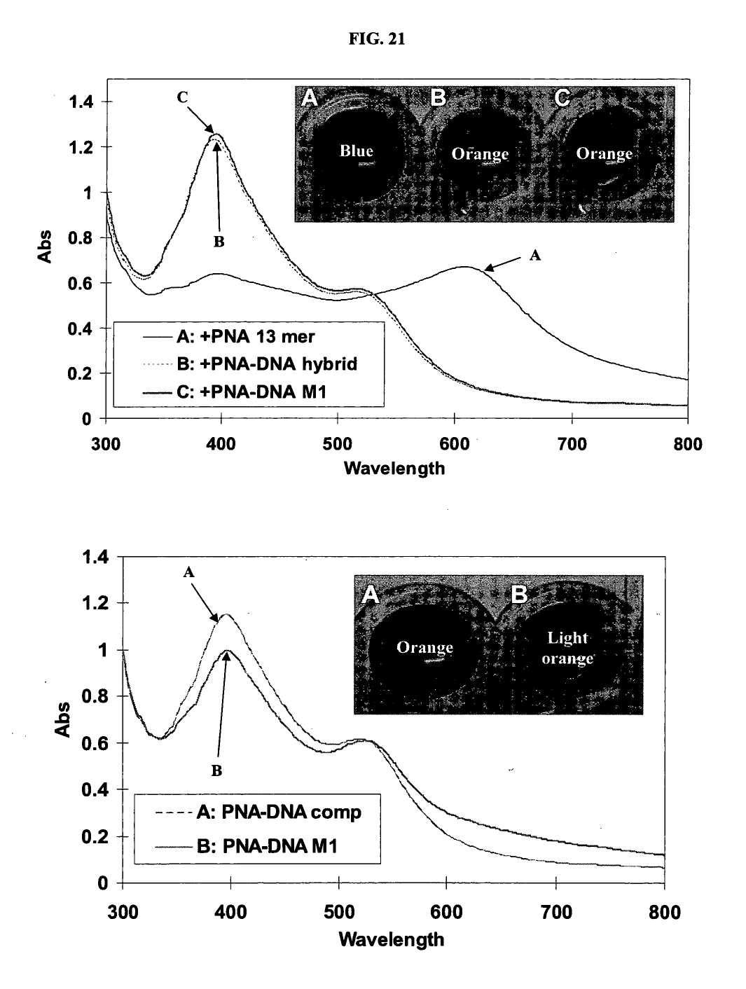

The discrimination between fully complementary DNA and SBM DNA is detected by the two-color-change method at 0.2 M NaCl (0.1 M NaCl was found less suitable to differentiate, FIG. 21, left panel). An A400 decrease (representative of aggregation of AgNPs) and a higher absorbance at longer wavelengths (representative of aggregation of AgNPs and AuNPs) were observed at the same time. These changes provide a parallel detection for the single-base-mismatch.

1. A method of detecting presence or absence of a target nucleic acid, wherein the method comprises:
providing a nucleic acid probe with a substantially neutral net charge and contacting the nucleic acid probe with a sample which is suspected to comprise the target nucleic acid to result in a first mixture; wherein the nucleic acid probe is substantially complementary to the target nucleic acid;
contacting the first mixture with negatively charged metallic nanoparticles to result in a second mixture;
determining whether the target nucleic acid is comprised in the second mixture or not.

2. A method of detecting presence or absence of a target nucleic acid, wherein the method comprises:
incubating negatively charged metallic nanoparticles with a sample which is suspected to comprise the target nucleic acid to form a first mixture;
contacting the first mixture with a nucleic acid probe with a substantially neutral net charge to result in a second mixture; wherein the nucleic acid probe is substantially complementary to the target nucleic acid;
determining whether the target nucleic acid is comprised in the second mixture or not.

3. The method of claim 1, wherein the nucleic acid probe with a substantially neutral net charge is a peptide nucleic acid.

4. The method of claim 1, wherein the nucleic acid probe comprises between about 8 to 50 bases.

5. The method of claim 4, wherein the nucleic acid probe comprises between about 10 to 30 bases.

6. The method of claim 1, wherein the negatively charged metallic nanoparticles are metallic particles with a negative surface charge.

7. The method of claim 1, wherein the metallic particles are noble metal nanoparticles.

8. The method of claim 7, wherein the noble metal nanoparticle are made of a noble metal selected from the group consisting of silver, gold and alloys of the aforementioned materials.

9. The method of claim 1, wherein the metallic nanoparticles each have a size in at least one dimension in a range of between about 10 nm to about 900 nm.

10. The method of claim 9, wherein the metallic nanoparticles each have a size in at least one dimension in a range of between about 10 nm to 50 nm.

11. The method of claim 1, wherein the metallic nanoparticles have a shape which is selected from the group consisting of a nanosphere, a nanocube, a nanorod, a nanotube, a nanostar, a nanocrescent, a nanoplate and mixtures thereof.

12. The method of claim 1, wherein the negative charge of the metallic nanoparticles is conferred by a carboxylic acid or sulfonic acid or carbolic acid or a mixture thereof immobilized at the surface of each of the metallic nanoparticles.

13. The method of claim 12, wherein the carboxylic acid is selected from the group consisting of citric acid, lactic acid, acetic acid, formic acid, oxalic acid, uric acid, pyrenedodecanoic acid, mercaptosuccinic acid, and aspartic acid.

14. The method of claim 1, wherein the target nucleic acid is comprised in an aqueous solution.

15. The method of claim 1, wherein the target nucleic acid is selected from the group consisting of DNA, RNA or derivatives thereof.

16. The method of claim 1, wherein the target nucleic acid is either fully complementary to the nucleic acid probe or comprises at least one base which is not complementary with the nucleic acid probe.

17. The method of claim 1, wherein the absence or presence of the target nucleic acid is determined by measuring the optical properties of the metallic nanoparticles or the size of the metallic nanoparticle aggregates, respectively.

18. The method of claim 1, wherein the amount of the target nucleic acid can be determined by correlating the optical properties of the metallic nanoparticles or the size of the metallic nanoparticle aggregates, respectively, in one sample with the optical properties or size of the metallic nanoparticle aggregates of another sample.

19. The method of claim 17, wherein the optical properties are determined with the naked eye or with a spectrophotometer.

20. The method of claim 17, wherein the size is determined with transmission electron microscopy (TEM) or light scattering techniques.

21. The method of claim 1, further comprising the addition of cations to the first mixture and/or second mixture.

22. The method of claim 21, wherein the salt comprises cations and anions.

23. The method of claim 22, wherein the concentration of the monovalent or divalent cations is between about 0.01 to about 0.5 M.

24. A kit for detecting presence or absence of a target nucleic acid in a sample; wherein the kit comprises:
at least one nucleic acid probe with a substantially neutral net charge; and
at least one type of negatively charged metallic nanoparticles.

25. Use of at least one type of negatively charged metallic particle in a method of detecting presence or absence of a target nucleic acid, wherein the method comprises:
providing a nucleic acid probe with a substantially neutral net charge and contacting the nucleic acid probe with a sample which is suspected to comprise the target nucleic acid to result in a first mixture; wherein the nucleic acid probe is substantially complementary to the target nucleic acid;
contacting the first mixture with negatively charged metallic nanoparticles to result in a second mixture;
determining whether the target nucleic acid is comprised in the second mixture or not.

26. Use of at least one nucleic acid probe with a substantially neutral net charge in a method of detecting presence or absence of a target nucleic acid, wherein the method comprises:
providing a nucleic acid probe with a substantially neutral net charge and contacting the nucleic acid probe with a sample which is suspected to comprise the target nucleic acid to result in a first mixture; wherein the nucleic acid probe is substantially complementary to the target nucleic acid;
contacting the first mixture with negatively charged metallic nanoparticles to result in a second mixture;
determining whether the target nucleic acid is comprised in the second mixture or not.
